(19) United States
(12) Patent Application Publication
(10) Pub. No.: US 2026/0357268 A1
(43) Pub. Date: Nov. 12, 2026

(54) COMPOSITIONS AND METHODS FOR TREATING CONDITIONS ASSOCIATED WITH UBE3A OVEREXPRESSION

(71) Applicant: Ultragenyx Pharmaceutical Inc., Novato, CA (US)

(72) Inventors: Andrzej Swistowski, Petaluma, CA (US); Scott Victor Dindot, College Station, TX (US); Julien Couthouis, Corte Madera, CA (US); Heather Richbourg Atwood, Novato, CA (US)

(21) Appl. No.: 19/705,373

(22) Filed: Jun. 11, 2026

Related U.S. Application Data
(63) Continuation of application No. PCT/US2024/059822, filed on Dec. 12, 2024.
(60) Provisional application No. 63/609,567, filed on Dec. 13, 2023.

(51) Int. Cl. C12N 15/113; A61P 25/00

(52) U.S. Cl. CPC C12N 15/1137; A61P 25/00; C12N 2310/11; C12N 2310/315; C12N 2310/321; C12N 2310/3231; C12N 2310/3341; C12N 2310/341

(57) ABSTRACT

This application provides antisense nucleic acid compounds that reduce UBE3A mRNA levels and/or UBE3A polypeptide levels in central nervous system neurons, thereby alleviating effects and symptoms caused by excess UBE3A levels. The application further provides compositions comprising antisense nucleic acid compounds and their use in methods of preventing or treating conditions associated with excess UBE3A mRNA and/or UBE3A polypeptide levels in a subject.

### CROSS-REFERENCE TO RELATED APPLICATIONS

This application is a continuation of International Application No. PCT/US2024/59822, filed on Dec. 12, 2024, which claims the benefit of and priority to U.S. Provisional Application No. 63/609,567, filed on Dec. 13, 2023, the entire disclosure of which is hereby incorporated herein by reference in its entirety for all purposes.

### REFERENCE TO A SEQUENCE LISTING

This application contains a Sequence Listing which has been submitted electronically in XML format. The Sequence Listing XML is incorporated herein by reference. Said XML file, created 25 Oct. 2024, is named UXN150-01WO_SL, and is 3,671,742 bytes in size.

### TECHNICAL FIELD OF THE DISCLOSURE

This disclosure relates to antisense nucleic acid compounds for inhibiting expression of ubiquitin protein ligase E3A (UBE3A) in neurons and compositions comprising the same for use in the treatment of conditions associated with UBE3A overexpression such as chromosome 15q11.2-q13.3 duplication syndrome (Dup15q syndrome).

### BACKGROUND OF THE INVENTION

Ubiquitin protein ligase E3A (UBE3A) poly-ubiquitinates cytoplasmic and nuclear proteins, targeting them for proteasomal degradation. This protein is expressed in many cell types, including in neurons, where it is highly localized to axon terminals, but it is also found in nuclei and mitochondria. In neurons, UBE3A is thought to locally regulate individual synapses through its activity in axon terminals and globally regulate neuronal physiology through its chromatin regulation activity in nuclei. UBE3A may be involved in the epigenetic regulation of neuronal imprinted genes by way of its regulation of DNA methylation and chromatin modeling. (See Lopez, Simon Jesse, David J. Segal, and Janine M. LaSalle. “UBE3A: An E3 ubiquitin ligase with genome-wide impact in neurodevelopmental disease.” Frontiers in Molecular Neuroscience 11 (2019): 476).

The ubiquitin protein ligase E3A (UBE3A) gene is located within the human 15q11.2-q13.3 locus, a unique and highly regulated genetic locus that is parentally imprinted in neuronal cells. Specifically, the maternal allele is selectively methylated whereas the paternal allele is non-methylated and expressed in neurons. At the same time, the paternal UBE3A allele is silenced in neurons by expression of the UBE3A anti-sense transcript (UBE3A-ATS). This unique regulation gives rise to three different human neurodevelopmental disorders stemming from mutations in the 15q11.2-q13.3 locus: deletion of the maternal allele gives rise to Angelman syndrome (AS) due to loss of UBE3A expression caused by silencing of the paternal allele; deletion of the paternal allele gives rise to Prader-Willi syndrome (PWS); and duplication of the maternal allele gives rise to chromosome 15q11.2-q13.3 duplication syndrome (Dup15q syndrome). (Lopez, et al., 2019; see also Copping, Nycole A., et al. “Neuronal overexpression of Ube3a isoform 2 causes behavioral impairments and neuroanatomical pathology relevant to 15q1.2-q13.3 duplication syndrome.” Human Molecular Genetics 26.20 (2017): 3995-4010).

Symptoms of Dup15q syndrome present with a high degree of diversity among patients, but frequently include hypotonia, speech/language disorder, developmental delay, behavior challenges, and abnormal EEG. Patients may also present with characteristic facial features, reduced growth, autism, sensory processing disorders, seizures, and hyperpigmentation. Dup15q syndrome patients usually have one of two forms of genetic disruption giving rise to the syndrome: maternal interstitial duplications and maternal isodicentric (“idic(15)”) duplications. Idic(15) patients are tetrasomic for the 15q11.2-q13 region (2 extra copies of the region, with 3 maternal copies and 1 paternal) and maternal interstitial duplication patients are triploid for the region (1 extra copy, with 2 maternal copies and 1 paternal). (See Kalsner, Louisa, and Stormy J. Chamberlain. “Prader-Willi, Angelman, and 15q11-q13 duplication syndromes.” Pediatric Clinics 62.3 (2015): 587-606).

Estimates vary, but it is believed that Dup15q syndrome affects between 1 in 5,000 to 1 in 20,000 individuals, yet no disease-modifying therapy exists. Others have described antisense oligonucleotides targeting human UBE3A (see United States Pat. App. Pub. No. US/2022/0259601A1 and Elamin, Marwa, et al. “The role of UBE3A in the autism and epilepsy-related Dup15q syndrome using patient-derived, CRISPR-corrected neurons.” Stem Cell Reports 18.4 (2023): 884-898); still, an effective ASO for treating Dup15q syndrome has yet to be identified. Thus, there remains a high unmet need for therapeutic approaches to treating Dup15q syndrome and other disorders arising from excess UBE3A expression or activity. The present disclosure addresses this need by providing novel nucleic acid molecules that target human UBE3A nucleic acids, including UBE3A genomic regions and transcripts, and reduce UBE3A expression in human neurons.

### BRIEF SUMMARY OF INVENTION

This disclosure provides compositions comprising novel nucleic acid molecules that can be used to inhibit or reduce expression of UBE3A in neurons. The disclosure further provides methods of using these compositions for the prevention or treatment of conditions associated with UBE3A overexpression in CNS neurons of a subject. More specifically, embodiments of this disclosure provide antisense nucleic acid compounds (i.e., “antisense compounds”) targeting a human UBE3A nucleic acid, and methods of their use for the treatment of Dup15q syndrome.

In one aspect, provided herein are antisense oligonucleotides (ASOs) comprising at least 16 contiguous nucleosides and having a nucleotide sequence selected from SEQ ID NO: 285 through SEQ ID NO: 562. The ASOs may comprise one or more of: a) a gap segment consisting of linked deoxynucleosides; b) a 5′ segment consisting of at least 2 linked nucleosides; c) a 3′ segment consisting of at least 2 linked nucleosides; d) at least one phosphorothioate internucleoside linkage; e) at least one nucleoside comprising a modified sugar; and f) at least one nucleoside comprising a modified nucleobase.

The gap segment of the ASOs may be positioned between the 5′ segment and the 3′ segment. The gap segment may comprise 5 to 15 linked nucleosides.

The 3′ segment of the ASOs may comprise 2-5 linked nucleosides.

The 5′ segment of the ASOs may comprise 2-5 linked nucleosides.

In some embodiments, at least one nucleoside of the 5′ segment of the ASOs and at least one nucleoside of the 3′ segment of the ASOs may comprise a modified sugar. In some embodiments, each nucleoside of the 5′ segment and each nucleoside of the 3′ segment comprises a modified sugar.

The modified sugar of the ASOs may comprise a bicyclic sugar. The bicyclic sugar may be selected from the group consisting of: 2′-O(CH2)2OCH3 (MOE); 4′-(CH2)-O-2′ (LNA); 4′-(CH2)2-O-2′ (ENA); and 4′-CH(CH3)-O-2′ (cEt).

In some embodiments, each internucleoside linkage of the ASOs is a phosphorothioate internucleoside linkage.

In some embodiments, a 5-methylcytosine nucleobase is substituted in place of a non-5-methyl cytosine residue in the ASOs.

The ASO may comprise a 3-10-3 LNA-DNA-LNA gapmer, wherein all internucleoside linkages are phosphorothioate internucleoside linkages.

The ASO may comprise a 3-11-3 LNA-DNA-LNA gapmer, wherein all internucleoside linkages are phosphorothioate internucleoside linkages.

The ASO may comprise a 3-12-3 LNA-DNA-LNA gapmer, wherein all internucleoside linkages are phosphorothioate internucleoside linkages.

The ASO may comprise a 4-11-5 MOE-DNA-MOE gapmer, wherein all internucleoside linkages are phosphorothioate internucleoside linkages.

The ASO may comprise a nucleotide sequence according to SEQ ID NO: 295, 307, 309, 310, 327, 328, 329, 330, 340, 341, 342, 343, 351, 356, 359, 366, 394, 402, 523, 527, 528, 529, 539, 542, 549, 550, 551, or 553.

In some embodiments, the ASO is a gapmer according to Compound ID No. 16-63326LNA, 17-29823LNA, 17-29855LNA, 17-29858LNA, 17-63264LNA, 17-63289LNA, 17-63290LNA, 17-63291LNA, 17-63324LNA, 17-63325LNA, 17-63326LNA, 17-63327LNA, 17-63458LNA, 17-67260LNA, 18-435LNA, 18-29854LNA, 18-63323LNA, 18-63458LNA, 17-63278LNA, 17-63286LNA, 17-63287LNA, 17-63288LNA, 17-63437LNA, 17-29853LNA, 18-63282LNA, 18-63285LNA, 18-63286LNA, or 18-63325LNA.

Also provided herein is an antisense oligonucleotide (ASO) comprising: a 5′ segment of 3 linked locked nucleic acid (LNA) nucleosides, a gap segment of linked deoxynucleosides, and a 3′ segment of 3 linked LNA nucleosides, wherein all internucleoside linkages are phosphorothioate internucleoside linkages, and wherein ASO comprises the nucleotide sequence of SEQ ID NO: 550.

Also provided herein is an antisense oligonucleotide (ASO) comprising: a 5′ segment of 3 linked locked nucleic acid (LNA) nucleosides, a gap segment of linked deoxynucleosides, and a 3′ segment of 3 linked LNA nucleosides, wherein all internucleoside linkages are phosphorothioate internucleoside linkages, and wherein ASO comprises the nucleotide sequence of SEQ ID NO: 542.

Also provided herein is an antisense oligonucleotide (ASO) comprising: a 5′ segment of 3 linked locked nucleic acid (LNA) nucleosides, a gap segment of linked deoxynucleosides, and a 3′ segment of 3 linked LNA nucleosides, wherein all internucleoside linkages are phosphorothioate internucleoside linkages, and wherein ASO comprises the nucleotide sequence of SEQ ID NO: 394.

Also provided herein is an antisense oligonucleotide (ASO) comprising: a 5′ segment of 3 linked locked nucleic acid (LNA) nucleosides, a gap segment of linked deoxynucleosides, and a 3′ segment of 3 linked LNA nucleosides, wherein all internucleoside linkages are phosphorothioate internucleoside linkages, and wherein ASO comprises the nucleotide sequence of SEQ ID NO: 328.

Also provided herein is an antisense oligonucleotide (ASO) comprising: a 5′ segment of 3 linked locked nucleic acid (LNA) nucleosides, a gap segment of linked deoxynucleosides, and a 3′ segment of 3 linked LNA nucleosides, wherein all internucleoside linkages are phosphorothioate internucleoside linkages, and wherein ASO comprises the nucleotide sequence of SEQ ID NO: 342.

Also provided herein is an antisense oligonucleotide (ASO) comprising: a 5′ segment of 3 linked locked nucleic acid (LNA) nucleosides, a gap segment of linked deoxynucleosides, and a 3′ segment of 3 linked LNA nucleosides, wherein all internucleoside linkages are phosphorothioate internucleoside linkages, and wherein ASO comprises the nucleotide sequence of SEQ ID NO: 356.

Also provided herein is an antisense oligonucleotide (ASO) comprising: a 5′ segment of 3 linked locked nucleic acid (LNA) nucleosides, a gap segment of linked deoxynucleosides, and a 3′ segment of 3 linked LNA nucleosides, wherein all internucleoside linkages are phosphorothioate internucleoside linkages, and wherein ASO comprises the nucleotide sequence of SEQ ID NO: 295.

Also provided herein is an antisense oligonucleotide (ASO) comprising: a 5′ segment of 3 linked locked nucleic acid (LNA) nucleosides, a gap segment of linked deoxynucleosides, and a 3′ segment of 3 linked LNA nucleosides, wherein all internucleoside linkages are phosphorothioate internucleoside linkages, and wherein ASO comprises the nucleotide sequence of SEQ ID NO: 351.

Also provided herein is an antisense oligonucleotide (ASO) comprising: a 5′ segment of 3 linked locked nucleic acid (LNA) nucleosides, a gap segment of linked deoxynucleosides, and a 3′ segment of 3 linked LNA nucleosides, wherein all internucleoside linkages are phosphorothioate internucleoside linkages, and wherein ASO comprises the nucleotide sequence of SEQ ID NO: 307.

In some embodiments, all cytosine nucleosides of the ASOs are replaced with 5-methylcytosine nucleosides.

In some embodiments, the ASOs comprise a 2′OMe-modified nucleoside at position 2 in the DNA gap.

Also provided herein are compositions comprising ASOs or salt thereof and a pharmaceutically acceptable carrier.

The composition may be a pharmaceutical formulation.

Also provided are methods of reducing the level of a UBE3A RNA in a cell, comprising contacting the cell with the ASO composition, thereby reducing the level of the UBE3A RNA in the cell.

Also provided are methods of inhibiting expression of UBE3A protein in a cell, comprising contacting the cell with the ASO or composition, thereby reducing expression of UBE3A protein in the cell.

In some embodiments, the cell is a central nervous system neuron.

In some embodiments, the cell is triploid or tetraploid for the UBE3A gene.

The cell may comprise a maternal duplication of the UBE3A allele.

The methods described herein may be performed on a cell in vitro. In some embodiments, the cell may be in a subject.

The subject may be a human and the ASO or composition may be administered to the subject.

In some embodiments, the ASO is administered to the subject via intravenous, subcutaneous, intramuscular, intraperitoneal, intrathecal, intra cisterna magna, or intraventricular (i.e., intracerebroventricular) injection.

Also provided are methods of treating, preventing, or ameliorating a disease associated with overexpression of UBE3A in central nervous system neurons in a subject, comprising administering to the subject an ASO or composition thereof described herein, thereby treating, preventing, or ameliorating the disease. The disease may be Dup15q syndrome. In some instances, the subject may have an idic(15) genotype.

In some embodiments, the methods may comprise administering to the subject a second pharmaceutical agent for treating, preventing, or ameliorating one or more symptoms of Dup15q syndrome. In some instances, the subject may have an idic(15) genotype.

In some embodiments, the ASOs or compositions thereof described herein are for use in a method of treating, preventing, or ameliorating a disease associated with overexpression of UBE3A in central nervous system neurons in a subject. The disease may be Dup15q syndrome. In some instances, the subject may have an idic(15) genotype.

Also provided are uses of an ASO or composition thereof described herein for the treatment of a disease associated with increased levels of UBE3A protein in central nervous system neurons in a subject. The disease may be Dup15q syndrome. In some instances, the subject may have an idic(15) genotype.

Also provided are uses of an ASO or composition thereof described herein for the preparation of a medicament for the treatment of a disease associated with increased levels of UBE3A protein in central nervous system neurons of a subject. The disease may be Dup15q syndrome. In some instances, the subject may have an idic(15) genotype.

### BRIEF DESCRIPTION OF DRAWINGS

FIG. 1 shows the dose response curve of ASO 18-63285LNA in normal human H9 neurons treated for 10 days as measured using multiplex QuantiGene assay.

FIG. 2 shows the dose response curve of ASO 17-29853LNA in normal human H9 neurons treated for 10 days as measured using multiplex QuantiGene assay.

FIG. 3 shows the dose response curve of ASO 18-63323LNA in normal human H9 neurons treated for 10 days as measured using multiplex QuantiGene assay.

FIG. 4 shows the dose response curve of ASO 17-67260LNA in normal human H9 neurons treated for 10 days as measured using multiplex QuantiGene assay.

FIG. 5 shows the dose response curve of ASO 16-63326LNA in normal human H9 neurons treated for 10 days as measured using multiplex QuantiGene assay.

FIG. 6 shows the dose response curve of ASO 17-63458LNA in normal human H9 neurons treated for 10 days as measured using multiplex QuantiGene assay.

FIG. 7 shows the dose response curve of ASO 17-29823LNA in normal human H9 neurons treated for 10 days as measured using multiplex QuantiGene assay.

FIG. 8 shows effects of 3 μM ASO treatment for 10 days on UBE3A mRNA levels in iPSC-derived Dup15q-idic(15) neurons (triplicates) compared to untreated controls.

FIG. 9 shows effects of 3 μM ASO treatment for 10 days on UBE3A protein levels in iPSC-derived Dup15q-idic(15) neurons compared to untreated controls.

FIG. 10 shows dose response curves of select ASOs (17-29853LNA and 17-29823LNA) treated for 10 days.

FIG. 11 shows dose-dependent UBE3A protein knockdown as confirmed by Western blot for ASO 17-29823LNA.

FIG. 12 shows in vivo target engagement of select ASOs in mouse brain (ASO1=18-63285LNA; ASO2=17-29853LNA; ASO3=18-63286LNA; ASO4=17-63286LNA; and ASO10=17-63458LNA).

### DETAILED DESCRIPTION OF THE INVENTION

This disclosure provides antisense nucleic acid compounds including, e.g., antisense oligonucleotides (ASOs), for use in therapeutic applications. In some embodiments, the antisense compounds including ASOs and compositions or formulations thereof of this disclosure can be used for ameliorating, preventing or treating conditions associated with excess UBE3A expression, including, e.g., chromosome 15q11.2-13.1 duplication syndrome, or Dup15q syndrome. Genetic and clinical evidence suggest that overexpression of the maternal UBE3A allele is the major contributor to the pathology observed in Dup15q syndrome, a neurodevelopmental disorder caused by maternal duplication of a genetic region on chromosome 15 called Prader-Willi Syndrome/Angelman Syndrome Critical Region (PWS/AS-CR). In CNS neurons, UBE3A is expressed only on the maternal chromosome as its expression on the paternal chromosome is epigenetically silenced. Full presentation of Dup15q syndrome occurs when UBE3A is overexpressed on the maternal chromosome. It is presently submitted that reduction of UBE3A overexpression in the CNS may ameliorate the neurological deficits observed in Dup15q syndrome individuals. Thus, antisense nucleic acid compounds are provided herein which are useful in reducing overexpression of UBE3A and/or normalizing UBE3A levels in Dup15q syndrome individuals.

In some embodiments, the antisense compounds of the disclosure reduce levels of a human UBE3A nucleic acid in a subject upon administration to the subject. In some embodiments, the antisense compounds of the disclosure reduce levels of UBE3A protein in a subject upon administration to the subject. The antisense compounds may reduce expression of UBE3A in a subject upon administration to the subject to levels seen under genetically normal conditions, i.e., with one paternal and one maternal copy present. In these manners, the antisense compounds provided herein may slow, ameliorate, or reverse the effects of UBE3A overexpression in neurons.

Thus, this disclosure encompasses synthetic, purified, antisense nucleic acid compounds such as antisense oligonucleotides (“ASOs”) for therapeutic use in reducing UBE3A nucleic acid and/or reducing UBE3A protein levels upon administration to a subject. The antisense compounds may contain natural and modified nucleotides, natural and modified internucleoside linkages, varying nucleotide lengths, and/or varying nucleoside (RNA and DNA) compositions, as provided herein.

### I. DEFINITIONS

Throughout the present specification, the terms “about” and/or “approximately” may be used in conjunction with numerical values and/or ranges. The term “about” is understood to mean those values near to a recited value. Furthermore, the phrases “less than about [a value]” or “greater than about [a value]” should be understood in view of the definition of the term “about” provided herein. The terms “about” and “approximately” may be used interchangeably. Unless otherwise noted, the term “about” when immediately preceding a numerical value means±10% of the numerical value.

Throughout the present specification, numerical ranges are provided for certain quantities. It is to be understood that these ranges comprise endpoints and all subranges therein, including each integer in and between a disclosed range. Thus, the range “from 50 to 80” includes all possible ranges therein (e.g., 51-79, 52-78, 53-77, 54-76, 55-75, 60-70, etc.) as well as each individual integer from 50 to 80 (e.g., 50, 51, 52, 53, 54, etc.). Where ranges are provided in the form of fractions, percentages, decimals, and the like, such ranges likewise include all possible subranges therein and each individual fraction, percentage, decimal, etc. in and between the disclosed range. For example, the range “from 0.1 to 1.0” includes all possible ranges therein (e.g., 0.2 to 0.9, etc.) and each individual 1/10th decimal from 0.1 to 1.0 (e.g., 0.1, 0.2, 0.3, 0.4, etc.). Furthermore, all values within a given range may be an endpoint for the range encompassed thereby (e.g., the range 50-80 includes the ranges with endpoints such as 55-80, 50-75, etc.).

The term “a” or “an” refers to one or more of that entity; for example, “an antisense oligonucleotide targeting UBE3A” refers to one or more such antisense oligonucleotides or at least one such antisense oligonucleotide. As such, the terms “a” (or “an”), “one or more” and “at least one” are used interchangeably herein.

The terms “comprise,” “comprising,” and the like, as used in this specification and in the claims are used in the non-limiting sense to mean that items following the word are included, but items not specifically mentioned are not excluded. The present invention may suitably “comprise”, “consist of”, or “consist essentially of”, the steps, elements, and/or reagents described in the claims.

“Antisense activity” means any detectable and/or measurable change attributable to the hybridization of an antisense compound to its target nucleic acid. Antisense activity includes knockdown or elimination of expression of a gene targeted by a compound having antisense activity.

“Antisense compound” means a compound comprising or consisting of an oligonucleotide at least a portion of which is complementary to a target nucleic acid to which it is capable of hybridizing, resulting in at least one antisense activity. An antisense oligonucleotide (ASO) is an “antisense compound”.

“Antisense oligonucleotide” or “ASO” means short (typically 12 to 25 nucleotides), single-stranded, synthetic DNA or RNA or hybrid DNA/RNA molecules designed to target either a coding or a non-coding nucleic acid such as an mRNA or other gene transcript sequence by complementary base-pairing in order to modulate its expression level.

“Bicyclic nucleoside” or “BNA” means a nucleoside comprising a bicyclic sugar moiety.

“Bicyclic sugar moiety” means a modified sugar moiety comprising a 4 to 7 membered ring (including but not limited to a furanosyl) comprising a bridge connecting two atoms of the 4 to 7 membered ring to form a second ring, resulting in a bicyclic structure. In some embodiments, the 4 to 7 membered ring is a sugar ring. In certain embodiments the 4 to 7 membered ring is a furanosyl. In certain such embodiments, the bridge connects the 2′-carbon and the 4′-carbon of the furanosyl.

“Ubiquitin protein ligase E3A” or “UBE3A” means any protein encoded by a UBE3A nucleic acid. The UBE3A gene gives rise to 33 known transcript variants. Each variant transcript and each processed mRNA arising from such variants may be a UBE3A nucleic acid, as used herein. In some embodiments, the UBE3A nucleic acid has the sequence set forth at RefSeqGene NG_009268.1 (human chromosome 15 (NC_000015.10) nucleotide positions 25,333,728 to U.S. Pat. No. 25,439,056 (genomic context)). In some embodiments, the UBE3A nucleic acid has the sequence set forth at GRCh38.p14 (hg38) chr15:25371667-25439024 (SEQ ID NO: 1) (referred to herein as the “UBE3A target region”). In some embodiments, the UBE3A nucleic acid has a sequence set forth in a variant transcript of the human UBE3A gene, including, e.g., RefSeq NM_000462.5, NM_001354505.1, NM_001354506.2, NM_001354507.2, NM_001354508.2, NM_001354509.2, NM_001354511.2, NM_001354512.2, NM_001354513.2, NM_001354523.2, NM_001354526.1, NM_001354538.2, NM_001354539.2, NM_001354540.2, NM_001354541.2, NM_001354542.2, NM_001354543.2, NM_001354544.2, NM_001354545.2, NM_001354546.2, NM_001354547.2, NM_001354548.2, NM_001354549.2, NM_001354550.2, NM_001354551.2, NM_001374461.1, NM_130838.4, or NM_130839.5. In some instances, throughout the specification, the term UBE3A is used as shorthand to refer to the UBE3A gene and/or the protein expressed from a UBE3A nucleic acid such as UBE3A mRNA.

“Complementary” in reference to oligomeric compounds (e.g., linked nucleosides, oligonucleotides, or nucleic acids) means the capacity of such oligomeric compounds or regions thereof to hybridize to another oligomeric compound or region thereof through nucleobase complementarity under stringent conditions. Complementary oligomeric compounds need not have nucleobase complementarity at each nucleoside. Rather, some mismatches are tolerated. In some embodiments, complementary oligomeric compounds or regions are complementary at 70% of the nucleobases (70% complementary). In certain embodiments, complementary oligomeric compounds or regions are 80% complementary. In certain embodiments, complementary oligomeric compounds or regions are 90% complementary. In certain embodiments, complementary oligomeric compounds or regions are 95% complementary. In certain embodiments, complementary oligomeric compounds or regions are 100% complementary.

“Conjugate” means a compound comprising two molecules that are covalently linked. In some embodiments, a conjugate comprises an antibody or other targeting agent and a modified oligonucleotide.

“Constrained ethyl nucleoside” or “cEt” means a nucleoside comprising a bicyclic sugar moiety comprising a 4′-CH(CH3)-O-2′ bridge.

“Expression” means the process by which a gene ultimately results in a protein. Expression includes, but is not limited to, transcription, post-transcriptional modification (e.g., splicing, polyadenylation, addition of 5′-cap), and translation.

“Hybridization” or “base-pairing” means the pairing of complementary oligomeric compounds (e.g., an antisense compound and its target nucleic acid). While not limited to a particular mechanism, the most common mechanism of pairing involves hydrogen bonding, which may be Watson-Crick, Hoogsteen or reversed Hoogsteen hydrogen bonding, between complementary nucleobases.

“Internucleoside linkage” means a covalent linkage between adjacent nucleosides in an oligonucleotide.

“Locked nucleic acid nucleoside” or “LNA” means a nucleoside comprising a bicyclic sugar moiety comprising a 4′-CH2-O-2′ bridge.

“Mismatch” means a nucleobase of a first oligomeric compound that is not capable of pairing with a nucleobase at a corresponding position of a second oligomeric compound, when the first and second oligomeric compound are aligned. Either or both of the first and second oligomeric compounds may be oligonucleotides.

“Modified internucleoside linkage” means any internucleoside linkage other than a naturally occurring internucleoside linkage.

“Modified nucleobases” include universal bases, hydrophobic bases, promiscuous bases, size-expanded bases, and fluorinated bases. Particular examples of modified nucleobases include 5-substituted pyrimidines, 6-azapyrimidines and N-2, N-6 and O-6 substituted purines, including 2-aminopropyladenine, 5-propynyluracil; 5-propynylcytosine; 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (C≡C—CH3) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine, 3-deazaguanine and 3-deazaadenine. Further modified nucleobases include tricyclic pyrimidines such as phenoxazine cytidine([5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified nucleobases may also include those in which the purine or pyrimidine base is replaced with other heterocycles, for example 7-deaza-adenine, 7-deazaguanosine, 2-aminopyridine and 2-pyridone.

“Modified nucleoside” means a nucleoside comprising at least one chemical modification compared to naturally occurring RNA or DNA nucleosides. Modified nucleosides may comprise a modified sugar moiety and/or a modified nucleobase.

“Modified oligonucleotide” means an oligonucleotide comprising at least one modified nucleoside and/or at least one modified internucleoside linkage. Examples of modified oligonucleotides include single-stranded and double-stranded compounds, such as, antisense oligonucleotides, siRNAs, shRNAs, ssRNAs, and occupancy-based compounds.

“Modified sugar” means a substituted sugar moiety or a sugar surrogate present in a modified nucleoside. Substituted sugar moieties include, but are not limited to furanosyls comprising substituents at the 2′-position, the 3′-position, the 5′-position, and/or the 4′-position. Certain substituted sugar moieties are bicyclic sugar moieties. Examples of modified sugars include 2′-substituted sugar moieties (a furanosyl comprising a substituent at the 2′-position other than H or OH). Sugar surrogates are structures that do not comprise a furanosyl but are capable of substituting for the naturally occurring sugar moiety of a nucleoside, such that the resulting nucleoside sub-units are capable of linking together and/or linking to other nucleosides to form an oligomeric compound which is capable of hybridizing to a complementary oligomeric compound. Such structures include rings comprising a different number of atoms than furanosyl (e.g., 4, 6, or 7-membered rings); replacement of the oxygen of a furanosyl with a non-oxygen atom (e.g., carbon, sulfur, or nitrogen); or both a change in the number of atoms and a replacement of the oxygen. Such structures may also comprise substitutions corresponding to those described for substituted sugar moieties (e.g., 6-membered carbocyclic bicyclic sugar surrogates optionally comprising additional substituents). Sugar surrogates also include more complex sugar replacements (e.g., the non-ring systems of peptide nucleic acid). Sugar surrogates include without limitation morpholinos, cyclohexenyls and cyclohexitols. Bicyclic sugar moieties comprise a 4 to 7 membered ring (including but not limited to a furanosyl) comprising a bridge connecting two atoms of the 4 to 7 membered ring to form a second ring, resulting in a bicyclic structure. In certain embodiments, the 4 to 7 membered ring is a sugar ring. In certain embodiments the 4 to 7 membered ring is a furanosyl. In certain such embodiments, the bridge connects the 2′-carbon and the 4′-carbon of the furanosyl.

“Modulation” means a change of amount or quality of a molecule, function, or activity following an intervention when compared to the amount or quality of the molecule, function, or activity prior to or without the intervention. For example, modulation includes the change, either an increase (stimulation or induction) or a decrease (inhibition or reduction) in gene expression. As a further example, modulation of expression can include a change in splice site selection of pre-mRNA processing, resulting in a change in the absolute or relative amount of a particular splice-variant compared to the amount in the absence of modulation.

“MOE” means —OCH2CH2OCH3.

“mRNA” means “messenger RNA”, i.e., an RNA molecule that encodes a protein.

“Naturally occurring internucleoside linkage” means a 3′ to 5′ phosphodiester linkage.

“Naturally occurring sugar moiety” means a ribofuranosyl as found in naturally occurring RNA or a deoxyribofuranosyl as found in naturally occurring DNA.

“Nucleobase” means a group of atoms that can be linked to a sugar moiety to create a nucleoside that is capable of incorporation into an oligonucleotide, and wherein the group of atoms is capable of bonding with a complementary naturally occurring nucleobase of another oligonucleotide or nucleic acid. Nucleobases may be naturally occurring or may be modified. “Unmodified nucleobase” or “naturally occurring nucleobase” means the naturally occurring heterocyclic nucleobases of RNA or DNA: the purine bases adenine (A) and guanine (G), and the pyrimidine bases thymine (T), cytosine (C) (including 5-methylcytosine), and uracil (U).

“Nucleoside” refers to a compound comprising a nucleobase moiety and a sugar moiety. Nucleosides include, but are not limited to, naturally occurring nucleosides (as found in DNA and RNA) and modified nucleosides. Nucleosides may be linked to a phosphate moiety.

“Nucleotide” means a nucleoside further comprising a phosphate linking group. “Linked nucleosides” may or may not be linked by phosphate linkages and thus include “linked nucleotides.” “Linked nucleosides” are nucleosides that are connected in a continuous sequence (i.e., no additional nucleosides are present between those that are linked).

“Oligomeric compound” means a polymeric structure comprising two or more sub-structures. In certain embodiments, an oligomeric compound comprises an oligonucleotide. In certain embodiments, an oligomeric compound consists of an oligonucleotide.

“Oligonucleotide” means a compound comprising a plurality of linked nucleosides. In certain embodiments, an oligonucleotide comprises one or more unmodified ribonucleosides (RNA) and/or unmodified deoxyribonucleosides (DNA) and/or one or more modified nucleosides.

“Pharmaceutically acceptable carrier or diluent” means any substance suitable for use in administering to an animal. In certain embodiments, a pharmaceutically acceptable carrier or diluent is sterile saline. In certain embodiments, such sterile saline is pharmaceutical grade saline.

“Polynucleotide” refers to an “oligomer,” i.e., a molecule comprising at least two monomers and includes oligonucleotides such as DNA oligonucleotides, RNA oligonucleotides, and mixed DNA/RNA oligonucleotides, as well as polynucleotides including DNA polynucleotides, RNA polynucleotides, and mixed DNA/RNA polynucleotides, as well as polynucleotides bearing synthetic or modified monomers such as modified nucleotides described herein.

“Pre-mRNA” means an RNA transcript that has not been fully processed into mRNA. Pre-RNA includes one or more intron.

“Subject” refers to a human or any non-human animal (e.g., mouse, rat, rabbit, dog, cat, cattle, swine, sheep, horse or primate). A human includes pre- and post-natal forms. In many embodiments, a subject is a human being. A subject can be a patient, which refers to a human presenting to a medical provider for diagnosis or treatment of a disease. The term “subject” is used herein interchangeably with “individual” or “patient” unless the context requires otherwise. A subject can be afflicted with or is susceptible to a disease or disorder but may or may not display symptoms of the disease or disorder.

“Substituted sugar moiety” means a furanosyl that is not a naturally occurring sugar moiety. Substituted sugar moieties include, but are not limited to furanosyls comprising substituents at the 2′-position, the 3′-position, the 5′-position and/or the 4′-position. Certain substituted sugar moieties are bicyclic sugar moieties.

“Sugar moiety” means a naturally occurring sugar moiety or a modified sugar moiety of a nucleoside.

“Sugar surrogate” means a structure that does not comprise a furanosyl and that is capable of replacing the naturally occurring sugar moiety of a nucleoside, such that the resulting nucleoside sub-units are capable of linking together and/or linking to other nucleosides to form an oligomeric compound which is capable of hybridizing to a complementary oligomeric compound. Such structures include rings comprising a different number of atoms than furanosyl (e.g., 4, 6, or 7-membered rings); replacement of the oxygen of a furanosyl with a non-oxygen atom (e.g., carbon, sulfur, or nitrogen); or both a change in the number of atoms and a replacement of the oxygen. Such structures may also comprise substitutions corresponding to those described for substituted sugar moieties (e.g., 6-membered carbocyclic bicyclic sugar surrogates optionally comprising additional substituents). Sugar surrogates also include more complex sugar replacements (e.g., the non-ring systems of peptide nucleic acid). Sugar surrogates include without limitation morpholinos, cyclohexenyls and cyclohexitols.

“Targeting” or “targeted to” means the association of an antisense compound to a particular sequence or region of a nucleic acid molecule. An antisense compound targets a target nucleic acid if it is sufficiently complementary to the target nucleic acid to allow hybridization under physiological conditions.

“2′-(ara)-F” refers to a 2′-F substituted nucleoside, wherein the fluoro group is in the arabino position.

“2′-deoxynucleoside” means a nucleoside comprising 2′-H furanosyl sugar moiety, as found in naturally occurring deoxyribonucleosides (DNA). In certain embodiments, a 2′-deoxynucleoside may comprise a modified nucleobase or may comprise an RNA nucleobase (e.g., uracil).

“2′-F nucleoside” refers to a nucleoside comprising a sugar comprising fluorine at the 2′ position. Unless otherwise indicated, the fluorine in a 2′-F nucleoside is in the ribo position (replacing the OH of a natural ribose).

“2′-substituted nucleoside” means a nucleoside comprising a substituent at the 2′-position other than H or OH. Unless otherwise indicated, a 2′-substituted nucleoside is not a bicyclic nucleoside.

“2′-substituted sugar moiety” means a furanosyl comprising a substituent at the 2′-position other than H or OH. Unless otherwise indicated, a 2′-substituted sugar moiety is not a bicyclic sugar moiety (i.e., the 2′-substituent of a 2′-substituted sugar moiety does not form a bridge to another atom of the furanosyl ring.

“3′-endo-furanosyl nucleoside” means an RNA-like nucleoside that comprises a substituted sugar moiety that has a 3′-endo conformation. 3′-endo-furanosyl nucleosides include, but are not limited to: 2′-MOE (“MOE”), 2′-F, 2′-OMe (“OMe”), locked nucleic acids (LNA), 2′-0,4′-C-ethylene-bridged nucleic acids (“ENA”), and 2′-O-Ethyl (cEt) nucleosides.

“Acyl,” means a radical formed by removal of a hydroxyl group from an organic acid and has the general Formula —C(O)—X where X is typically aliphatic, alicyclic or aromatic. Examples include aliphatic carbonyls, aromatic carbonyls, aliphatic sulfonyls, aromatic sulfinyls, aliphatic sulfinyls, aromatic phosphates, aliphatic phosphates and the like. Acyl groups as used herein may optionally include further substituent groups.

“Alicyclic” means a cyclic ring system wherein the ring is aliphatic. The ring system can comprise one or more rings wherein at least one ring is aliphatic. Preferred alicyclics include rings having from about 5 to about 9 carbon atoms in the ring. Alicyclic as used herein may optionally include further substituent groups.

“Aliphatic” means a straight or branched hydrocarbon radical containing up to 24 carbon atoms wherein the saturation between any two carbon atoms is a single, double or triple bond. An aliphatic group preferably contains from 1 to about 24 carbon atoms, more typically from 1 to about 12 carbon atoms with from 1 to about 6 carbon atoms being more preferred. The straight or branched chain of an aliphatic group may be interrupted with one or more heteroatoms that include nitrogen, oxygen, sulfur and phosphorus. Such aliphatic groups interrupted by heteroatoms include without limitation, polyalkoxys, such as polyalkylene glycols, polyamines, and polyimines Aliphatic groups as used herein may optionally include further substituent groups.

“Alkenyl,” means a straight or branched hydrocarbon chain radical containing up to 24 carbon atoms and having at least one carbon-carbon double bond. Examples of alkenyl groups include without limitation, ethenyl, propenyl, butenyl, 1-methyl-2-buten-1-yl, dienes such as 1,3-butadiene and the like. Alkenyl groups typically include from 2 to about 24 carbon atoms, more typically from 2 to about 12 carbon atoms with from 2 to about 6 carbon atoms being more preferred. Alkenyl groups as used herein may optionally include one or more further substituent groups.

“Alkoxy” means a radical formed between an alkyl group and an oxygen atom wherein the oxygen atom is used to attach the alkoxy group to a parent molecule. Examples of alkoxy groups include without limitation, methoxy, ethoxy, propoxy, isopropoxy, n-butoxy, sec-butoxy, tert-butoxy, n-pentoxy, neopentoxy, n-hexoxy and the like. Alkoxy groups as used herein may optionally include further substituent groups.

“Alkyl,” means a saturated straight or branched hydrocarbon radical containing up to 24 carbon atoms. Examples of alkyl groups include without limitation, methyl, ethyl, propyl, butyl, isopropyl, n-hexyl, octyl, decyl, dodecyl and the like. Alkyl groups typically include from 1 to about 24 carbon atoms, more typically from 1 to about 12 carbon atoms (C1-C12 alkyl) with from 1 to about 6 carbon atoms being more preferred.

“Alkynyl,” means a straight or branched hydrocarbon radical containing up to 24 carbon atoms and having at least one carbon-carbon triple bond. Examples of alkynyl groups include, without limitation, ethynyl, 1-propynyl, 1-butynyl, and the like. Alkynyl groups typically include from 2 to about 24 carbon atoms, more typically from 2 to about 12 carbon atoms with from 2 to about 6 carbon atoms being more preferred. Alkynyl groups as used herein may optionally include one or more further substituent groups.

“Aminoalkyl” means an amino substituted C1-C12 alkyl radical. The alkyl portion of the radical forms a covalent bond with a parent molecule. The amino group can be located at any position and the aminoalkyl group can be substituted with a further substituent group at the alkyl and/or amino portions.

“Aralkyl” and “arylalkyl” mean an aromatic group that is covalently linked to a C1-C12 alkyl radical. The alkyl radical portion of the resulting aralkyl (or arylalkyl) group forms a covalent bond with a parent molecule. Examples include without limitation, benzyl, phenethyl and the like. Aralkyl groups as used herein may optionally include further substituent groups attached to the alkyl, the aryl or both groups that form the radical group.

“Aryl” and “aromatic” mean a mono- or polycyclic carbocyclic ring system radicals having one or more aromatic rings. Examples of aryl groups include without limitation, phenyl, naphthyl, tetrahydronaphthyl, indanyl, idenyl and the like. Preferred aryl ring systems have from about 5 to about 20 carbon atoms in one or more rings. Aryl groups as used herein may optionally include further substituent groups.

“Furanosyl” means a structure comprising a 5-membered ring comprising four carbon atoms and one oxygen atom.

“Halo” and “halogen,” mean an atom selected from fluorine, chlorine, bromine and iodine.

“Heteroaryl,” and “heteroaromatic,” mean a radical comprising a mono- or polycyclic aromatic ring, ring system or fused ring system wherein at least one of the rings is aromatic and includes one or more heteroatoms. Heteroaryl is also meant to include fused ring systems including systems where one or more of the fused rings contain no heteroatoms. Heteroaryl groups typically include one ring atom selected from sulfur, nitrogen or oxygen. Examples of heteroaryl groups include without limitation, pyridinyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, benzimidazolyl, benzooxazolyl, quinoxalinyl and the like. Heteroaryl radicals can be attached to a parent molecule directly or through a linking moiety such as an aliphatic group or hetero atom. Heteroaryl groups as used herein may optionally include further substituent groups.

### II. OVERVIEW

The antisense compounds targeting human UBE3A nucleic acids described herein and compositions or formulations comprising the same may be used to ameliorate, prevent, or treat any symptom or disorder associated with excess UBE3A expression in a subject, such as symptoms or disorders arising from a maternal duplication within the 15q11.2-q13.3 region, e.g., Dup15q syndrome. For example, the antisense compounds targeting human UBE3A nucleic acids may be used to ameliorate, prevent, or treat any symptom or disease associated with excess UBE3A protein in cells, e.g., neurons, of a subject. In some embodiments, the subject may present with Dup15q syndrome.

The antisense compounds targeting human UBE3A nucleic acids described herein may be any antisense compound useful for inhibiting expression of the target gene, i.e., UBE3A. Example sequences for portions of antisense compounds targeting human UBE3A nucleic acids are provided in Table 1. For example, antisense compounds include single and double stranded nucleic acid compounds and may suppress target gene expression via RNaseH-mediated mRNA decay, steric hindrance-based mechanisms, RNA-induced silencing complex (RISC), or guide strand-directed nuclease-mediated mRNA decay mechanisms. Thus, antisense compounds targeting human UBE3A nucleic acids described herein may comprise antisense oligonucleotides (ASOs), RNAi compounds such as small interfering RNA (siRNA) and short hairpin RNA (shRNA), or nuclease guide RNA (gRNA) compounds for use with nucleases such as zinc finger nucleases (ZFNs), transcription activator-like effector nucleases (TALENs), Cas9, and similar nucleases. In general, persons skilled in the art are aware of modifications needed to adapt the antisense compounds disclosed herein, such as those in Table 1, to be suitable for use as, e.g., an ASO, an siRNA, an shRNA, or a gRNA. Such modifications include, e.g., incorporation of sense and/or antisense strands, preparation of single or double stranded compositions, use of DNA or RNA nucleosides, and oligonucleotide length, among others.

In some embodiments, the antisense compounds of the disclosure are antisense oligonucleotides or “ASOs”. The ASOs of the disclosure are designed to target human UBE3A nucleic acids, such as UBE3A mRNA, and thereby modulate its expression. The ASOs can be administered to a subject in need (e.g., a patient with Dup15q syndrome) and reduce functionally active UBE3A mRNA or UBE3A protein levels in the subject. Thus, the ASOs described herein and compositions or formulations comprising the same can be used for preventing, treating, ameliorating, or reversing any symptoms of UBE3A overexpression in a subject. In an exemplary embodiment, the subject is a human patient presenting with a maternal duplication within the 15q11.2-q13.3 region, resulting in excess expression of UBE3A protein in neurons. In some embodiments, a composition or formulation comprising an ASO of the disclosure can be delivered to the central nervous system of the subject.

### II. ANTISENSE COMPOUNDS TARGETING HUMAN UBE3A TRANSCRIPT

In certain embodiments, the present invention provides antisense nucleic acid compounds targeting human UBE3A nucleic acids (including mRNA transcripts of the UBE3A gene) to suppress expression of the UBE3A gene. The antisense compounds may comprise oligonucleotides. In some embodiments, oligonucleotides of the disclosure comprise one or more chemical modifications such as one or more nucleoside modification (including modifications to the sugar moiety and/or the nucleobase) and/or modifications to one or more internucleoside linkage.

### A. Modified Nucleosides

The antisense compounds of the disclosure may comprise or consist of oligonucleotides comprising at least one modified nucleoside. Such modified nucleosides may comprise a modified sugar moiety, a modified nucleobase, or both a modified sugar moiety and a modified nucleobase.

i. Sugar Modifications

The antisense compounds described herein may comprise one or more nucleosides comprising a modified sugar to confer desirable properties, such as enhanced nuclease stability or increased binding affinity with a target nucleic acid relative to antisense compounds comprising non-modified sugar moieties. Modified sugars may be substituted sugar moieties, bicyclic or tricyclic sugar moieties, or sugar surrogates.

Substituted sugar moieties may comprise one or more substituent, including but not limited to substituents at the 2′ and/or 5′ positions. Examples of sugar substituents suitable for the 2′-position include, but are not limited to: 2′-F, 2′-OCH3 (“OMe” or “O-methyl”), and 2′-O(CH2)2OCH3 (“MOE”). In certain embodiments, sugar substituents at the 2′ position are selected from allyl, amino, azido, thio, 0-allyl substitutions. Examples of sugar substituents at the 5′-position, include, but are not limited to: 5′-methyl (R or S); 5′-vinyl, 5′-vinylphosphonate, and 5′-methoxy.

Nucleosides comprising 2′-substituted sugar moieties are referred to as 2′-substituted nucleosides. In certain embodiments, a 2′-substituted nucleoside comprises a 2′-substituent group selected from halo, allyl, amino, azido, O—C1-C10 alkoxy; O—C1-C10 substituted alkoxy, SH, CN, OCN, CF3, OCF3, O-alkyl, S-alkyl, N(Rm)-alkyl; O-alkenyl, S-alkenyl, or N(Rm)-alkenyl; O-alkynyl, S-alkynyl, N(Rm)-alkynyl; O-alkylenyl-O-alkyl, alkynyl, alkaryl, aralkyl, O-alkaryl, O-aralkyl, O(CH2)2SCH3, O—(CH2)2-O—N(Rm)(Rn) or O—CH2-C(═O)—N(Rm)(Rn), where each Rm and Rn is, independently, H, an amino protecting group or substituted or unsubstituted C1-C10 alkyl.

In certain embodiments, a 2′-substituted nucleoside comprises a 2′-substituent group selected from F, NH2, N3, OCF3, O—CH3, O(CH2)3NH2, CH2-CH═CH2, O—CH2 CH═CH2, OCH2CH2OCH3, O(CH2)2SCH3, O—(CH2)2-O—N(Rm)(Rn), O(CH2)2O(CH2)2N(CH3)2, and N-substituted acetamide (O—CH2-C(═O)—N(Rm)(Rn) where each Rm and Rn is, independently, H, an amino protecting group or substituted or unsubstituted C1-C10 alkyl.

Certain modified sugar moieties comprise a bridging sugar substituent that forms a second ring resulting in a bicyclic sugar moiety. In certain such embodiments, the bicyclic sugar moiety comprises a bridge between the 4′ and the 2′ furanose ring atoms. Examples of such 4′ to 2′ sugar substituents, include, but are not limited to: [C(Ra)(Rb)]n, [C(Ra)(Rb)]n-O—, C(RaRb)—N(R)—O or, C(RaRb)—O—N(R)—; 4′-CH2-2′, 4′-(CH2)2-2′,4′-(CH2)3-2′, 4′-(CH2)-O-2′ (LNA); 4′-(CH2)-S-2; 4′-(CH2)2-O-2′ (ENA); 4′-CH(CH3)-O-2′ (cEt) and 4′-CH(CH2OCH3)-O-2′, and analogs thereof (see, e.g., U.S. Pat. No. 7,399,845, issued on Jul. 15, 2008); 4′-C(CH3)(CH3)-O-2′ and analogs thereof, (see, e.g., WO/2009/006478); 4′-CH2-N(OCH3)-2′ and analogs thereof (see, e.g., WO/2008/150729); 4′-CH2-O—N(CH3)-2′ (see, e.g., US/2004/0171570); 4′-CH2-O—N(R)-2′, and 4′-CH2-N(R)—O-2′-, wherein each R is, independently, H, a protecting group, or C1-C12 alkyl; 4′-CH2-N(R)—O-2′, wherein R is H, C1-C12 alkyl, or a protecting group (see, U.S. Pat. No. 7,427,672, issued on Sep. 23, 2008); 4′-CH2-C(H)(CH3)-2′ (see, e.g., Chattopadhyaya, et al., J. Org. Chem., 2009, 74, 118-134); and 4′-CH2-C(═CH2)-2′ and analogs thereof (see WO/2008/154401).

Nucleosides comprising bicyclic sugar moieties are referred to as bicyclic nucleosides or BNAs. Bicyclic nucleosides include, but are not limited to, (A) α-L-Methyleneoxy (4′-CH2-O-2′) BNA, (B) β-D-Methyleneoxy (4′-CH2-O-2′) BNA (also referred to as locked nucleic acid or LNA), (C) Ethyleneoxy (4′-(CH2)2-O-2′) BNA, (D) Aminooxy (4′-CH2-O N(R)-2′) BNA, (E) Oxyamino (4′-CH2-N(R)O-2′) BNA, (F) Methyl(methyleneoxy) (4′-CH(CH3)-O-2′) BNA (also referred to as constrained ethyl or cEt), (G) methylene-thio (4′-CH2-S-2′) BNA, (H) methylene-amino (4′-CH2-N(R)-2′) BNA, (I) methyl carbocyclic (4′-CH2-CH(CH3)-2′) BNA, (J) propylene carbocyclic (4′-(CH2)3-2′) BNA, and (M) 4′-CH2-O—CH2-2′.

Additional bicyclic sugar moieties are known in the art, for example: Singh et al., Chem. Commun., 1998, 4, 455-456; Koshkin et al., Tetrahedron, 1998, 54, 3607-3630; Wahlestedt et al., Proc. Natl. Acad. Sci. U.S.A., 2000, 97, 5633-5638; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 129(26) 8362-8379 (Jul. 4, 2007); Elayadi et al., Curr. Opinion Invens. Drugs, 2001, 2, 558-561; Braasch et al., Chem. Biol., 2001, 8, 1-7; Orum et al., Curr. Opinion Mol. Ther., 2001, 3, 239-243; U.S. Pat. Nos. 7,053,207, 6,268,490, 6,770,748, 6,794,499, 7,034,133, 6,525,191, 6,670,461, and 7,399,845; WO/2004/106356, WO/1994/14226, WO/2005/021570, and WO/2007/134181; U.S. Patent Application Publication Nos. US/2004/0171570, US/2007/0287831, and US/2008/0039618; and WO/2008/150729A2, WO/2008/154401A2, and WO/2009/006478A2.

In certain embodiments, bicyclic sugar moieties and nucleosides incorporating such bicyclic sugar moieties are further defined by isomeric configuration. For example, a nucleoside comprising a 4′-2′ methylene-oxy bridge, may be in the α-L configuration or in the β-D configuration. Previously, α-L-methyleneoxy (4′-CH2-O-2′) bicyclic nucleosides have been incorporated into antisense oligonucleotides that showed antisense activity (Frieden et al., Nucleic Acids Research, 2003, 21, 6365-6372).

In certain embodiments, substituted sugar moieties comprise one or more non-bridging sugar substituent and one or more bridging sugar substituent (e.g., 5′-substituted and 4′-2′ bridged sugars). (See WO/2007/134181, wherein LNA is substituted with, for example, a 5′-methyl or a 5′-vinyl group).

In certain embodiments, modified sugar moieties are sugar surrogates. In certain such embodiments, the oxygen atom of the naturally occurring sugar is substituted, e.g., with a sulfur, carbon or nitrogen atom. In certain such embodiments, such modified sugar moiety also comprises bridging and/or non-bridging substituents as described above. For example, certain sugar surrogates comprise a 4′-sulfur atom and a substitution at the 2′-position (see, e.g., U.S. Patent Application Publication US/2005/0130923) and/or the 5′ position.

In certain embodiments, sugar surrogates comprise rings having other than 5-atoms. For example, in certain embodiments, a sugar surrogate comprises a six-membered tetrahydropyran. Such tetrahydropyrans may be further modified or substituted. Nucleosides comprising such modified tetrahydropyrans include, but are not limited to, hexitol nucleic acid (HNA), anitol nucleic acid (ANA), mannitol nucleic acid (MNA) (see Leumann, C J. Bioorg. &Med. Chem. (2002) 10:841-854), and fluoro HNA (F-HNA).

Many other bicyclic and tricyclic sugar and sugar surrogate ring systems are known in the art that can be used to modify nucleosides (see, e.g., review article: Leumann, J. C, Bioorganic &Medicinal Chemistry, 2002, 10, 841-854).

In certain embodiments, sugar surrogates comprise rings having more than 5 atoms and more than one heteroatom. For example, nucleosides comprising morpholino sugar moieties and their use in antisense compounds has been reported (see for example: Braasch et al., Biochemistry, 2002, 41, 4503-4510; and U.S. Pat. Nos. 5,698,685; 5,166,315; 5,185,444; and 5,034,506).

In certain embodiments, morpholinos may be modified, for example by adding or altering various substituent groups from the above morpholino structure. Such sugar surrogates are referred to herein as “modified morpholinos.”

Combinations of modifications are also provided without limitation, such as 2′-F-5′-methyl substituted nucleosides (see WO/2008/101157 for other disclosed 5′,2′-bis substituted nucleosides) and replacement of the ribosyl ring oxygen atom with S and further substitution at the 2′-position (see U.S. Patent Application Publication US/20050130923) or alternatively 5′-substitution of a bicyclic nucleic acid (see WO/2007/134181, wherein a 4′-CH2-O-2′ bicyclic nucleoside is further substituted at the 5′ position with a 5′-methyl or a 5′-vinyl group). The synthesis and preparation of carbocyclic bicyclic nucleosides along with their oligomerization and biochemical studies have also been described (see, e.g., Srivastava et al., J. Am. Chem. Soc. 2007, 129(26), 8362-8379).

ii. Modified Nucleobases

The antisense compounds described herein may comprise one or more nucleosides comprising modified nucleobases.

Modified nucleobases may include universal bases, hydrophobic bases, promiscuous bases, size-expanded bases, and fluorinated bases. Particular examples of modified nucleobases include 5-substituted pyrimidines, 6-azapyrimidines and N-2, N-6 and O-6 substituted purines, including 2-aminopropyladenine, 5-propynyluracil; 5-propynylcytosine; 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (C≡C-CH3) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine, 3-deazaguanine and 3-deazaadenine. Further modified nucleobases include tricyclic pyrimidines such as phenoxazine cytidine([5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified nucleobases may also include those in which the purine or pyrimidine base is replaced with other heterocycles, for example 7-deaza-adenine, 7-deazaguanosine, 2-aminopyridine and 2-pyridone.

### B. Modified Internucleoside Linkages

Individual nucleosides of the antisense compounds described herein may be linked together using any internucleoside linkage to form oligonucleotides. Internucleoside linkages include phosphorus containing internucleoside linkages including, but not limited to, phosphodiesters (P═O), phosphotriesters, methylphosphonates, phosphoramidates, and phosphorothioates (P═S). Non-phosphorus containing internucleoside linkages include, but are not limited to, methylenemethylimino (—CH2-N(CH3)-O—CH2-), thiodiester (—O—C(O)—S—), thionocarbamate (—O—C(O)(NH)—S—); siloxane (—O—Si(H)2-O—); and N,N′-dimethylhydrazine (—CH2-N(CH3)-N(CH3)-). Modified linkages, compared to natural phosphodiester linkages, may improve nuclease resistance of the oligonucleotide. In some embodiments, internucleoside linkages having a chiral atom can be prepared as a racemic mixture, or as separate enantiomers. Representative chiral linkages include, but are not limited to, alkylphosphonates and phosphorothioates. Methods of preparing phosphorous-containing and non-phosphorous-containing internucleoside linkages are known to those skilled in the art.

### C. Oligonucleotide Motifs

Antisense compounds described herein may comprise oligonucleotides having one or more chemical modification, i.e., one or more modified sugars and/or one or more modified nucleobases and/or one or more modified internucleoside linkages. These chemical modifications (sugar modifications, nucleobase modifications, and/or linkage modifications) may be present in a given oligonucleotide according to a defined pattern or motif. The patterns of chemical modifications of sugar moieties, internucleoside linkages, and nucleobases may be independent of one another. Thus, an oligonucleotide may be described by its sugar modification motif, internucleoside linkage motif and/or nucleobase modification motif (independent of the sequence of nucleobases).

i. Sugar Motifs

Oligonucleotides described herein may comprise one or more type of modified sugar moieties and/or naturally occurring sugar moieties arranged along an oligonucleotide or region thereof in a defined pattern.

For example, the oligonucleotides may comprise or consist of a region having a gapmer sugar motif, which comprises two external regions or segments and a central or internal region or gap. The three regions of a gapmer sugar motif (the 5′-segment, the gap segment, and the 3′-segment) form a contiguous sequence of nucleosides wherein at least some of the sugar moieties of the nucleosides of each of the 5′ and 3′ segments differ from at least some of the sugar moieties of the nucleosides of the gap. Specifically, at least the sugar moieties of the nucleosides of each 5′ and 3′ segment that are closest to the gap (the 3′-most nucleoside of the 5′-segment and the 5′-most nucleoside of the 3′-segment) differ from the sugar moiety of the neighboring gap nucleosides, thus defining the boundary between the 3′ and 5′ segments and the gap. In some embodiments, the sugar moieties within the gap are the same as one another. In some embodiments, the gap includes one or more nucleoside having a sugar moiety that differs from the sugar moiety of one or more other nucleosides of the gap. In some embodiments, the sugar motifs of the 5′ and the 3′ segments are the same as one another (symmetric sugar gapmer). In certain embodiments, the sugar motifs of the 5′-segment differs from the sugar motif of the 3′-segment (asymmetric sugar gapmer).

Segments of a gapmer may be different lengths or equal lengths. For example, in some instances, a gapmer consists of a 5 nucleoside 5′ segment, a 5 nucleoside gap segment, and a 5 nucleoside 3′ segment. In other instances, a gapmer consists of, e.g., a 3 nucleoside 5′ segment, a 9 nucleoside gap segment, and a 3 nucleoside 3′ segment. In still other instances, a gapmer consists of, e.g., a 2 nucleoside 5′ segment, a 10 nucleoside gap segment, and a 3 nucleoside 3′ segment. Thus, each of the segments may be of differing lengths compared to the other segments. The lengths of 5′ and 3′ segments can vary, typically from 2 nucleosides to 5 nucleosides in length. The length of the gap segment can also vary, typically from 5 nucleosides to 15 nucleosides in length.

Gapmers can be referred to according to their segment lengths in the 5′ to 3′ direction. For instance, a gapmer consisting of a 3 nucleoside 5′ segment, a 10 nucleoside gap, and a 3 nucleoside 3′ segment can be referred to as a 3-10-3 gapmer, more specifically a 3-10-3 16mer. For specific gapmers where sugar modifications are defined, the gapmers can be more specifically referred to by defining the sugar modifications of each segment. For example, a gapmer consisting of a 3 nucleoside 5′ segment having 4′-CH2-2′, 4′-(CH2)2-2′,4′-(CH2)3-2′, or 4′-(CH2)-O-2′ (LNA) sugar modifications, a gap having 10 linked deoxynucleosides, and a 3 nucleoside 3′ segment having 2′-O(CH2)2OCH3 (“MOE”) sugar modifications can be referred to as a 3-10-3 LNA-DNA-MOE gapmer, more specifically a 3-10-3 16mer.

Gapmers may comprise one or more modified sugar at a specific position in one or more segment. That is, gapmers may comprise a sugar modification at a specific nucleoside position within the 5′ segment, the gap segment, and/or the 3′ segment. In some embodiments, gapmers described herein comprise a 2′OMe modified sugar at a specific position within the DNA gap. For example, gapmers may comprise a 2′OMe modified sugar at the nucleoside of position 2 in the DNA gap.

ii. Nucleobase Modification Motifs

Oligonucleotides described herein may comprise chemical modifications to nucleobases arranged along the oligonucleotide or region/segment thereof in a defined pattern or nucleobase modification motif. In certain embodiments, each nucleobase is modified.

Oligonucleotides may comprise a block of modified nucleobases. In certain such embodiments, the block is at the 3′-end of the oligonucleotide. In certain embodiments the block is within 3 nucleotides of the 3′-end of the oligonucleotide. In certain such embodiments, the block is at the 5′-end of the oligonucleotide. In certain embodiments the block is within 3 nucleotides of the 5′-end of the oligonucleotide.

Nucleobase modifications can depend on the base at a particular position of an oligonucleotide. For example, in certain embodiments each purine or each pyrimidine in an oligonucleotide is modified. In certain embodiments, each adenine is modified. In certain embodiments, each guanine is modified. In certain embodiments, each thymine is modified. In certain embodiments, each cytosine is modified. In certain embodiments, each uracil is modified.

In some embodiments, oligonucleotides comprise one or more nucleosides comprising a modified nucleobase. In certain embodiments, oligonucleotides having a gapmer sugar motif comprise a nucleoside comprising a modified nucleobase. In certain such embodiments, one nucleoside comprising a modified nucleobase is in the central gap of an oligonucleotide having a gapmer sugar motif. In certain embodiments, the sugar is an unmodified 2′ deoxynucleoside. In certain embodiments, the modified nucleobase is selected from: a 2-thio pyrimidine and a 5-propyne pyrimidine.

In some embodiments, some, all, or none of the cytosine moieties in an oligonucleotide are 5-methylcytosine. 5-methylcytosine is not a “modified nucleobase.” Accordingly, unless otherwise indicated, unmodified nucleobases include both cytosine residues having a 5-methyl and those lacking a 5-methyl. In some embodiments, the methylation state of all or some cytosine nucleobases is specified.

### D. Length of Antisense Compounds

The present invention provides antisense nucleic acid compounds including oligonucleotides of differing nucleotide lengths. For example, the antisense compounds may consist of 8 to 9, 8 to 10, 8 to 11, 8 to 12, 8 to 13, 8 to 14, 8 to 15, 8 to 16, 8 to 17, 8 to 18, 8 to 19, 8 to 20, 8 to 21, 8 to 22, 8 to 23, 8 to 24, 9 to 10, 9 to 11, 9 to 12, 9 to 13, 9 to 14, 9 to 15, 9 to 16, 9 to 17, 9 to 18, 9 to 19, 9 to 20, 9 to 21, 9 to 22, 9 to 23, 9 to 24, 9 to 25, 10 to 11, 10 to 12, 10 to 13, 10 to 14, 10 to 15, 10 to 16, 10 to 17, 10 to 18, 10 to 19, 10 to 20, 10 to 21, 10 to 22, 10 to 23, 10 to 24, 10 to 25, 11 to 12, 11 to 13, 11 to 14, 11 to 15, 11 to 16, 11 to 17, 11 to 18, 11 to 19, 11 to 20, 11 to 21, 11 to 22, 11 to 23, 11 to 24, 11 to 25, 12 to 13, 12 to 14, 12 to 15, 12 to 16, 12 to 17, 12 to 18, 12 to 19, 12 to 20, 12 to 21, 12 to 22, 12 to 23, 12 to 24, 12 to 25, 13 to 14, 13 to 15, 13 to 16, 13 to 17, 13 to 18, 13 to 19, 13 to 20, 13 to 21, 13 to 22, 13 to 23, 13 to 24, 13 to 25, 14 to 15, 14 to 16, 14 to 17, 14 to 18, 14 to 19, 14 to 20, 14 to 21, 14 to 22, 14 to 23, 14 to 24, 14 to 25, 15 to 16, 15 to 17, 15 to 18, 15 to 19, 15 to 20, 15 to 21, 15 to 22, 15 to 23, 15 to 24, 15 to 25, 16 to 17, 16 to 18, 16 to 19, 16 to 20, 16 to 21, 16 to 22, 16 to 23, 16 to 24, 16 to 25, 17 to 18, 17 to 19, 17 to 20, 17 to 21, 17 to 22, 17 to 23, 17 to 24, 17 to 25, 18 to 19, 18 to 20, 18 to 21, 18 to 22, 18 to 23, 18 to 24, 18 to 25, 19 to 20, 19 to 21, 19 to 22, 19 to 23, 19 to 24, 19 to 25, 20 to 21, 20 to 22, 20 to 23, 20 to 24, 20 to 25, 21 to 22, 21 to 23, 21 to 24, 21 to 25, 22 to 23, 22 to 24, 22 to 25, 23 to 24, 23 to 25 or 24 to 25 linked nucleosides. In some embodiments, an antisense oligonucleotide of the disclosure can be from about 12 nucleotides to about 25 nucleotides in length, such as 12, 13, 14, 15, 16, 17, 18, 19, 20, 21, 22, 23, 24, or 25 nucleotides in length. Generally, oligonucleotides having a length of n linked nucleosides are referred to as “nmers” such that an oligonucleotide of 16 linked nucleosides is referred to as a 16mer.

### E. Conjugations for Improved Biodistribution

Antisense compounds described herein may be modified by attachment of one or more conjugate groups. In general, conjugate groups modify one or more properties of the attached antisense compound including but not limited to pharmacodynamics, pharmacokinetics, stability, binding, absorption, cellular distribution, cellular uptake, biodistribution, charge, and clearance. Conjugate groups are used in the chemical arts and are linked directly or via an optional conjugate linking moiety or conjugate linking group to a parent compound such as an antisense compound, such as an oligonucleotide. Conjugate groups include without limitation, intercalators, reporter molecules, polyamines, polyamides, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins and dyes.

In some embodiments, conjugate groups are attached to oligonucleotides by a conjugate linking group. In certain such embodiments, conjugate linking groups, including, but not limited to, bifunctional linking moieties such as those known in the art are amenable to the compounds provided herein. Conjugate linking groups are useful for attachment of conjugate groups, such as chemical stabilizing groups, functional groups, reporter groups and other groups to selective sites in a parent compound such as for example an antisense compound. In general, a bifunctional linking moiety comprises a hydrocarbyl moiety having two functional groups. One of the functional groups is selected to bind to a parent molecule or compound of interest and the other is selected to bind essentially any selected group such as chemical functional group or a conjugate group. In some embodiments, the conjugate linker comprises a chain structure or an oligomer of repeating units such as ethylene glycol or amino acid units. Examples of functional groups that are routinely used in a bifunctional linking moiety include, but are not limited to, electrophiles for reacting with nucleophilic groups and nucleophiles for reacting with electrophilic groups. In some embodiments, bifunctional linking moieties include amino, hydroxyl, carboxylic acid, thiol, unsaturations (e.g., double or triple bonds), and the like.

Some nonlimiting examples of conjugate linking moieties include pyrrolidine, 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other linking groups include, but are not limited to, substituted C1-C10 alkyl, substituted or unsubstituted C2-C10 alkenyl or substituted or unsubstituted C2-C10 alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

Conjugate groups may be attached to either or both ends (that is, 5′ and/or 3′ ends) of an oligonucleotide (terminal conjugate groups) and/or at any internal position. In some embodiments, conjugate groups are at the 5′-end of an antisense compound described herein, or near the 5′-end. In some embodiments, conjugate groups are at the 3′-end of an antisense compound described herein, or near the 3′-end.

In some embodiments, conjugate groups may interact with the lipid components of the cell membrane, bind to specific cell surface proteins or receptors, and/or penetrate the cell through endogenous transport mechanisms carrying the antisense compound with them.

The conjugate can be attached at the 5′- and/or the 3′-end of a sense and/or antisense strand of an antisense compound via a covalent attachment such as a nucleic acid or non-nucleic acid linker. The conjugate can be attached through a carbamate group or other linking group (see, e.g., U.S. Patent Application Publication Nos. US/2005/0074771, US/2005/0043219, and US/2005/0158727).

In some embodiments, the conjugate group may be a molecular entity that facilitates the delivery of the antisense compound into a cell or may be a molecule that comprises a drug or label. Examples of conjugate molecules suitable for attachment to antisense compounds of the present disclosure include, without limitation, lipophilic molecules (e.g., fatty acids), cholesterols, glycols such as polyethylene glycol (PEG), human serum albumin (HSA), carotenoids, terpenes, bile acids, folates (e.g., folic acid, folate analogs and derivatives thereof), sugars (e.g., galactose, galactosamine, N-acetyl galactosamine, glucose, mannose, fructose, fucose, etc.), phospholipids, peptides, ligands for cellular receptors capable of mediating cellular uptake, antibodies, aptamers, and combinations thereof (see, e.g., U.S. Patent Application Publication Nos. US/2003/0130186, US/2004/0110296, and US/2004/0249178; and U.S. Pat. No. 6,753,423).

The conjugate group used and the extent of conjugation to the antisense compound can be evaluated for improved pharmacokinetic profiles, bioavailability, biodistribution, and/or stability of the antisense compound. As such, one skilled in the art can screen antisense compounds having various conjugates attached thereto to identify conjugates having improved properties using any of a variety of well-known in vitro cell culture techniques or in vivo animal models.

In some embodiments, an antisense compound disclosed herein may be conjugated to a lipophilic molecule (for example, a long chain fatty acid or LCFA), an antibody (for example, anti-transferrin receptor antibody), an aptamer, a ligand, a peptide, or a polymer.

In some embodiments, an antisense compound disclosed herein may be conjugated to a ligand or receptor having specific binding activity to a cognate receptor or ligand of a neuron. Thus, the antisense compounds may be specifically targeted to neurons by binding receptors or ligands expressed thereon.

In some embodiments, an antisense compound disclosed herein may be conjugated to an antibody. In some embodiments, the antibody is a neuron-targeting antibody, e.g., an antibody having specificity for a neuron-specific cell surface marker.

### IV. PHARMACEUTICAL FORMULATIONS

In some aspects, this application provides compositions containing antisense nucleic acid compounds described herein and a suitable carrier such as a buffer, solvent, or diluent. The compositions may be liquid, wherein the antisense compound is dissolved in a suitable solvent or diluent, or may be solid or lyophilized, wherein the antisense compound is present in dry form with a suitable carrier or buffer. Such compositions containing antisense compounds can further be formulated for particular uses, such as for achieving a pharmacological effect in a cell based system and/or for pharmaceutical application. Thus, the disclosure provides pharmaceutical formulations containing a polynucleotide such as an ASO of the disclosure and a pharmaceutically acceptable carrier.

A pharmaceutical formulation can be capable of local or systemic administration. In some aspects, a pharmaceutical formulation can be capable of any mode of administration. In certain aspects, the administration can be by any route, including intravenous, intrathecal, intracerebroventricular, subcutaneous, pulmonary, intramuscular, intraperitoneal, dermal, oral, inhalation or nasal administration.

The present disclosure provides pharmaceutical formulations comprising one or more antisense compound described herein. Such pharmaceutical formulations may comprise a suitable pharmaceutically acceptable diluent or carrier, such as a pharmaceutical grade diluent or carrier. In some embodiments, a pharmaceutical formulation comprises a sterile saline solution and one or more antisense compound. In certain embodiments, such pharmaceutical formulations may consist of a sterile saline solution and one or more antisense compound. In some embodiments, a pharmaceutical formulation comprises one or more antisense compound and sterile water. In certain embodiments, a pharmaceutical formulation consists of one or more antisense compound and sterile water. In some embodiments, a pharmaceutical formulation comprises one or more antisense compound and phosphate-buffered saline (PBS).

The antisense compounds of the disclosure may be admixed with pharmaceutically acceptable active and/or inert substances for the preparation of pharmaceutical formulations or formulations depending on intended route of administration, extent of disease, or dose to be administered.

Pharmaceutical formulations provided herein comprising antisense compounds encompass any pharmaceutically acceptable salts, esters, or salts of such esters. In certain embodiments, pharmaceutical formulations comprising antisense compounds comprise one or more oligonucleotide which, upon administration to an animal, including a human, is capable of providing (directly or indirectly) the biologically active metabolite or residue thereof. Accordingly, for example, the disclosure is also drawn to pharmaceutically acceptable salts of antisense compounds, prodrugs, pharmaceutically acceptable salts of such prodrugs, and other bioequivalents. Suitable pharmaceutically acceptable salts include, but are not limited to, sodium and potassium salts.

A prodrug can include the incorporation of additional nucleosides at one or both ends of an antisense compound which are cleaved by endogenous nucleases within the body, to form the active antisense nucleic acid compound.

In certain embodiments, a pharmaceutical formulation provided herein comprises a delivery system. Examples of delivery systems include, but are not limited to, liposomes and emulsions. Certain delivery systems are useful for preparing certain pharmaceutical formulations including those comprising hydrophobic compounds. In certain embodiments, certain organic solvents such as dimethylsulfoxide are used.

Lipid moieties have been used in nucleic acid therapies and delivery systems therefor in a variety of compositions and methods. For example, the nucleic acid may be introduced into preformed liposomes or lipoplexes made of mixtures of cationic lipids and neutral lipids. In certain methods, DNA complexes with mono- or poly-cationic lipids are formed without the presence of a neutral lipid. In certain embodiments, a lipid moiety is selected to increase distribution of a pharmaceutical agent to a particular cell or tissue. In certain embodiments, a lipid moiety is selected to increase distribution of a pharmaceutical agent to fat tissue. In certain embodiments, a lipid moiety is selected to increase distribution of a pharmaceutical agent to muscle tissue.

In some embodiments, an antisense compound may be delivered via liposomes, nanoparticles, lipid nanoparticles (LNPs), polymers, microparticles, microcapsules, micelles, or extracellular vesicles and formulations comprising the same.

In some embodiments, an antisense compound may be delivered via a lipid nanoparticle (LNP). Examples of LNPs capable of delivering antisense compounds are described in WO/2015/074085, WO/2016/081029, WO/2017/117530, WO/2018/118102, WO/2018/119163, WO/2018/222926, WO/2019/191780, and WO/2020/154746. In some embodiments, an LNP may be decorated with targeting moiety, e.g., an antibody, a receptor, or a fragment thereof capable of binding to a target ligand.

In some embodiments, a lipid nanoparticle comprises (a) a nucleic acid (e.g., an antisense compound described herein), (b) a cationic lipid, (c) an aggregation reducing agent (such as a PEG-lipid), (d) optionally a non-cationic lipid (such as a neutral lipid), and (e) optionally a sterol. In one embodiment, the lipid nanoparticle comprises (i) at least one cationic lipid; (ii) a neutral lipid, e.g., DSPC; (iii) a sterol, e.g., cholesterol; and (iv) a PEG-lipid, in a molar ratio of about 20-65% cationic lipid: 5-25% neutral lipid: 25-55% sterol; 0.5-15% PEG-lipid. In some embodiments, the cationic lipid is selected from ATX-002, ATX-081, ATX-095, or ATX-126, as described in WO/2018/222926.

In some embodiments, an antisense compound is delivered via a nanocarrier comprising a molecule enabling specific receptor-mediated endosomal uptake. In one embodiment, said molecule can enable receptor binding, endosomal uptake, controlled breakdown of the endosomal membrane, and release of the antisense compound into a target cell. Examples of nanocarriers capable of delivering antisense compounds are described in WO/2009/141257. In some embodiments, the nanocarrier is a lipid-based nanocarrier, e.g., a lipid nanoparticle (LNP).

Pharmaceutical formulations provided herein may comprise one or more modified oligonucleotides and one or more excipients. In certain such embodiments, excipients are selected from water, salt solutions, alcohol, polyethylene glycols, gelatin, lactose, amylase, magnesium stearate, talc, silicic acid, viscous paraffin, hydroxymethylcellulose, and polyvinylpyrrolidone.

Pharmaceutical formulations provided herein may comprise a co-solvent system comprising, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol comprising 3% w/v benzyl alcohol, 8% w/v of the nonpolar surfactant Polysorbate 80™ and 65% w/v polyethylene glycol 300. The proportions of such co-solvent systems may be varied considerably without significantly altering their solubility and toxicity characteristics. Furthermore, the identity of co-solvent components may be varied; for example: other surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose.

Pharmaceutical formulations as described herein may be formulated for, i.e., prepared for purposes of, oral administration, nasal administration, buccal administration, or administration by injection (e.g., intravenous, intrathecal, intracerebroventricular, subcutaneous, intramuscular, etc.). For example, a pharmaceutical formulation may comprise a carrier and be formulated in aqueous solution, such as water or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical formulations for injection are presented in unit dosage form, e.g., in ampoules or in multi-dose containers. Certain pharmaceutical formulations for injection are suspensions, solutions or emulsions in oily or aqueous vehicles, and may contain formulatory agents such as suspending, stabilizing and/or dispersing agents. Certain solvents suitable for use in pharmaceutical formulations for injection include, but are not limited to, lipophilic solvents and fatty oils, such as sesame oil, synthetic fatty acid esters, such as ethyl oleate or triglycerides, and liposomes.

### V. METHODS OF ADMINISTRATION

An effective dose of an antisense compound or pharmaceutical formulation comprising an antisense compound of this disclosure can be an amount that is sufficient to reduce the amount of UBE3A mRNA or UBE3A protein in a cell or tissue that expresses the UBE3A gene under physiological conditions. For example, an effective dose of the antisense compounds provided herein may reduce the level of UBE3A mRNA expressed in a neuron of a Dup15q syndrome individual. In some instances, the effective dose reduces the level of UBE3A expression from the levels seen in a tetraploid idic(15) individual to the levels seen in a diploid individual. In some instances, the effective dose reduces the level of UBE3A expression from the levels seen in a triploid Dup15q syndrome individual to the levels seen in a diploid individual. In these ways, an effective dose may normalize the level of UBE3A expression in neurons of a Dup15q individual relative to a subject having no maternal duplication in the UBE3A allele. An effective dose of an antisense compound or pharmaceutical formulation comprising an antisense compound of this disclosure reduces the level of UBE3A mRNA or UBE3A protein expressed in a central nervous system neuron by about 10%, 20%, 30%, 40%, 50%, 60%, 70%, 80%, 90%, or more compared to a neuron that expresses the UBE3A gene under physiological conditions in the absence of the effective dose of an antisense compound or pharmaceutical formulation thereof described herein. In some embodiments, an effective dose reduces the amount of UBE3A mRNA or UBE3A protein by ⅓ or by about ½. In some embodiments, the effective dose reduces the amount of UBE3A mRNA or UBE3A protein by more than ½.

A therapeutically effective dose can be an amount of an antisense compound or formulation thereof that is sufficient to reduce, slow, halt, or reverse the effects of UBE3A overexpression in central nervous system neuron of a subject having Dup15q syndrome. In some embodiments, a therapeutically effective dose reduces the amount of UBE3A mRNA or UBE3A protein by about 10%, 20%, 30%, 40%, 50%, 60%, 70%, 80%, 90%, or more compared to neurons of the subject absent the therapeutically effective dose of an antisense compound or pharmaceutical formulation thereof described herein. In some embodiments, a therapeutically effective dose of the antisense compounds or formulations thereof provided herein normalize UBE3A expression levels in the central nervous system of a Dup15q syndrome individual to levels seen in an individual who is diploid for the UBE3A allele, i.e., does not have a maternal duplication in the allele.

A therapeutically effective dose can be administered to a subject in one or more separate administrations, and by different routes. As will be appreciated in the art, a therapeutically effective dose or a therapeutically effective amount is largely determined based on the total amount of the therapeutic agent contained in the pharmaceutical formulations of the present disclosure. Generally, a therapeutically effective amount is sufficient to achieve a meaningful benefit to the subject (e.g., treating, modulating, curing, preventing and/or ameliorating a disease or condition associated with UBE3A overexpression in central nervous system neurons). For example, a therapeutically effective amount may be an amount sufficient to achieve a desired therapeutic and/or prophylactic effect. Generally, the amount of a therapeutic agent (e.g., an ASO described herein) administered to a subject in need thereof will depend upon the characteristics of the subject. Such characteristics include ploidy of the UBE3A allele, disease severity, general health, age, sex and body weight of the subject. One of ordinary skill in the art armed with the present disclosure may be able to determine appropriate dosages depending on these and other related factors such as pharmacodynamic and pharmacokinetic properties of the therapeutic agents described herein. In addition, both objective and subjective assays may optionally be employed to identify optimal dosage ranges.

Therapeutically effective doses of the antisense compounds described herein can be administered to subjects by appropriate administration routes. For example, doses may be administered via local or systemic administration. In some aspects, doses of the antisense compounds are administered to a subject via intravenous, intrathecal, intracerebroventricular, subcutaneous, pulmonary, intramuscular, intraperitoneal, dermal, oral, inhalation or nasal administration. In some embodiments, therapeutically effective doses of the antisense compounds are administered directly to the central nervous system of a subject. CNS delivery routes suitable for administration of the antisense compounds and pharmaceutical formulations thereof described herein include, e.g., intrathecal injection, intra cisterna magna injection, and intraventricular (intracerebroventricular) injection. In some embodiments, the compounds and formulations described herein are administered by intrathecal injection into the cerebrospinal fluid (CSF) of a subject in a reclined, e.g., Trendelenburg, position with or without a post-injection fluid flush to disperse the injected fluid and increase CNS distribution in the subject.

Methods provided herein contemplate single as well as multiple administrations of a therapeutically effective amount of the polynucleotide (e.g., an ASO targeting UBE3A nucleic acids) described herein. Pharmaceutical formulations comprising a polynucleotide described herein can be administered at regular intervals, depending on the nature, severity and extent of the subject's condition (e.g., the severity of a subject's disease state and the associated symptoms). In some embodiments, a therapeutically effective amount of the polynucleotide of the present disclosure may be administered periodically at regular intervals, e.g., once every year, once every six months, once every four months, once every three months, once every two months, once a month, once every two weeks, once a week, or more frequently than once a week. For example, a therapeutically effective amount of an ASO of the present disclosure may be administered weekly, once every two weeks, or monthly.

In some embodiments, the pharmaceutical formulations of the present disclosure are formulated such that they are suitable for extended-release of the polynucleotide contained therein. Such extended-release compositions may be conveniently administered to a subject at extended dosing intervals. Also contemplated herein are pharmaceutical formulations which are formulated for depot administration (e.g., subcutaneously, intramuscularly) to either deliver or release a polynucleotide of the disclosure over extended periods of time. The extended-release means employed may be combined with modifications made to the polynucleotide to enhance stability.

In some embodiments, administering a therapeutically effective dose of a composition or formulation comprising a polynucleotide of the disclosure can result in decreased levels of UBE3A protein in CNS neurons of a treated subject. In some embodiments, administering a composition or formulation comprising a polynucleotide of the disclosure results in a ⅓, ½, ⅓ to ½, or greater than ½ decrease in levels of UBE3A protein in neurons of the subject relative to a baseline UBE3A protein level in the subject prior to treatment or relative to a baseline U1BE3A protein level in age-matched or disease-state-matched or genotype-matched subjects having Dup15q syndrome. In certain embodiments, administering a therapeutically effective dose of a composition or formulation comprising a polynucleotide of the disclosure will result in a decrease in levels of UBE3A protein relative to baseline UBE3A protein levels in CNS tissues of the subject prior to treatment.

In some embodiments, a therapeutically effective dose, when administered regularly, results in reduced expression of UBE3A in CNS neurons as compared to baseline levels prior to treatment. In some embodiments, administering a therapeutically effective dose of a compound or formulation comprising an antisense compound of the disclosure results in the expression of UBE3A in CNS neurons of the subject at a level at or below about ⅓ or ½, or at or below about 75%, about 70%, about 65%, about 60%, about 55%, about 50%, about 45%, about 40%, about 35%, about 30%, or about 25% compared to expression in neurons of the subject prior to treatment.

A therapeutically effective dose of an antisense compound described herein or formulation thereof may comprise about 0.01 to about 50 mg of the antisense compound per kg body weight of the subject. For instance, the therapeutically effective dose may be about 0.1 to about 50 mg/kg body weight, about 0.1-25 mg/kg, about 0.1-20 mg/kg, about 0.1-15 mg/kg, about 0.1-10 mg/kg, about 0.1-5 mg/kg, about 0.1-1 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 1-50 mg/kg, about 5-50 mg/kg, about 10-50 mg/kg, about 15-50 mg/kg, about 20-50 mg/kg, or about 25-50 mg/kg body weight of the subject.

In some embodiments, a therapeutically effective dose of an antisense compound described herein or formulation thereof may comprise about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, or about 20 mg of the antisense compound. The therapeutically effective dose of an antisense compound may be based on the subject's age, sex, disease severity, UBE3A copy number, or a combination thereof.

### VI. METHODS OF TREATMENT

The present disclosure provides antisense compounds and formulations thereof for use in ameliorating, preventing, delaying onset of, or treating conditions associated with increased expression of UBE3A in central nervous system neurons in a subject, such as symptoms or disorders arising from a maternal duplication in the UBE3A gene of a subject. Thus, also provided are methods for ameliorating, preventing, delaying onset of, or treating conditions associated with increased UBE3A protein levels in the CNS of a subject, such as symptoms or disorders arising from a maternal duplication in the UBE3A gene of a subject. In some embodiments, the subject is diagnosed with Dup15q syndrome. In some embodiments, the subject is diagnosed with isodicentric duplication Dup15q syndrome (idic(15)). In some embodiments, the methods comprise administering to the subject an antisense compound or a pharmaceutical formulation thereof. In some embodiments, the methods may further comprise the administration of one or more additional therapeutics (i.e., a “second pharmaceutical agents”) before, simultaneous with, or after administration of the antisense compound or pharmaceutical formulation thereof.

Pharmaceutical agents that may be co-administered as a second pharmaceutical agent according to the methods described herein include, e.g., growth hormones, seizure medications (such as lorazepam, diazepam, rufinamide, phenytoin, valproic acid, felbamate, levetiracetam, lamotrigine, clobazam, carbamazepine, topiramate, oxcarbazepine, lacosamide, zonisamide, prednisone/prednisolone, and vigabatrin) medications for abnormal behaviors (such as clonidine, sertraline, risperidone, quetiapine, and olanzapine) and antidepressants.

Throughout the description, where compositions are described as having, including, or comprising specific components, or where processes and methods are described as having, including, or comprising specific steps, it is contemplated that, additionally, there are compositions of the present disclosure that consist essentially of, or consist of, the recited components, and that there are processes and methods according to the present disclosure that consist essentially of, or consist of, the recited steps.

In the application, where an element or component is said to be included in and/or selected from a list of recited elements or components, it will be understood that the element or component can be any one of the recited elements or components, or the element or component can be selected from a group consisting of two or more of the recited elements or components.

Further, it will be understood that elements and/or features of a composition or a method described herein can be combined in a variety of ways without departing from the spirit and scope of the present disclosure, whether explicit or implicit herein. For example, where reference is made to a particular compound, that compound can be used in various embodiments of compositions of the present disclosure and/or in methods of the present disclosure, unless otherwise understood from the context. In other words, within this application, embodiments have been described and depicted in a way that enables a clear and concise application to be written and drawn, but it is intended and will be appreciated that embodiments may be variously combined or separated without parting from the present teachings and disclosure(s). For example, it will be appreciated that all features described and depicted herein can be applicable to all aspects of the disclosure(s) described and depicted herein. All of the features disclosed in this specification may be combined in any combination. Each feature disclosed in this specification may be replaced by an alternative feature serving the same, equivalent, or similar purpose.

It will be understood that the expression “at least one of” includes individually each of the recited objects after the expression and the various combinations of two or more of the recited objects unless otherwise understood from the context and use. The expression “and/or” in connection with three or more recited objects will be understood to have the same meaning unless otherwise understood from the context.

The use of the term “include,” “includes,” “including,” “have,” “has,” “having,” “contain,” “contains,” or “containing,” including grammatical equivalents thereof, will be understood generally as open-ended and non-limiting, for example, not excluding additional unrecited elements or steps, unless otherwise specifically stated or understood from the context.

The use of any and all examples, or exemplary language herein, for example, “for instance”, “such as”, “for example”, “e.g.,”, or “including” is intended merely to illustrate better the present disclosure and does not pose a limitation on the scope of the disclosure unless claimed. No language in the specification will be construed as indicating any non-claimed element as essential to the practice of the subject matter of the present disclosure.

It is understood that this disclosure is not limited to the particular methodology, protocols, materials, and reagents described, as these may vary. It is also to be understood that the terminology used herein is for the purpose of describing particular embodiments only, and is not intended to limit the scope of the present disclosure, which will be encompassed by the appended claims.

While antisense nucleic acid compounds, compositions, formulations, and methods described herein have been described with specificity according to certain embodiments, the following examples serve only to illustrate examples of these compounds, compositions, formulations, and methods, and are not intended to limit the same.

Although the sequence listing accompanying this filing identifies each sequence as either “RNA” or “DNA” as required, in reality, those sequences may be modified with any combination of chemical modifications. One of skill in the art will readily appreciate that such designation as “RNA” or “DNA” to describe modified oligonucleotides is, in certain instances, arbitrary. For example, an oligonucleotide comprising a nucleoside comprising a 2′-OH sugar moiety and a thymine base could be described as a DNA having a modified sugar (2′-OH for the natural 2′-H of DNA) or as an RNA having a modified base (thymine(methylated uracil) for natural uracil of RNA).

Accordingly, nucleic acid sequences provided herein, including, but not limited to those in the sequence listing, are intended to encompass nucleic acids containing any combination of natural or modified RNA and/or DNA, including, but not limited to such nucleic acids having modified nucleobases. By way of further example and without limitation, an antisense nucleic acid compound having the nucleobase sequence “ATCGATCG” encompasses any nucleic acid compounds having such nucleobase sequence, whether modified or unmodified, including, but not limited to, such compounds comprising RNA bases, such as those having sequence “AUCGAUCG” and those having some DNA bases and some RNA bases such as “AUCGATCG” and nucleic acid compounds having other modified or naturally occurring bases, such as “ATmeCGAUCG,” wherein meC indicates a cytosine base comprising a methyl group at the 5-position.

All publications, patents and patent applications, including any drawings, sequences, and appendices therein referred to throughout the present description are incorporated by reference in their entirety for all purposes to the same extent as if each individual publication, patent or patent application, drawing, sequence, or appendix was specifically and individually indicated to be incorporated by reference in its entirety for all purposes.

### ENUMERATED EMBODIMENTS

The scope of this disclosure is indicated by the appended claims and the following enumerated embodiments, and all changes that come within the meaning and range of equivalency of the claims and enumerated embodiments are intended to be embraced therein. Thus, the disclosure provides:

Embodiment 1. A nucleic acid compound, such as an antisense oligonucleotide (ASO), comprising at least 16 contiguous nucleosides and having a nucleotide sequence selected from SEQ ID NO: 285 through SEQ ID NO: 562, comprising one or more of:
a) a gap segment consisting of linked deoxynucleosides;
b) a 5′ segment consisting of at least 2 linked nucleosides;
c) a 3′ segment consisting of at least 2 linked nucleosides;
d) at least one phosphorothioate internucleoside linkage;
e) at least one nucleoside comprising a modified sugar; and
f) at least one nucleoside comprising a modified nucleobase.

Embodiment 2. The nucleic acid compound of Embodiment 1, wherein the gap segment is positioned between the 5′ segment and the 3′ segment.

Embodiment 3. The nucleic acid compound of Embodiment 1 or 2, wherein the gap segment comprises 5 to 15 linked nucleosides.

Embodiment 4. The nucleic acid compound of any one of Embodiments 1-3, wherein the 3′ segment comprises 2-5 linked nucleosides.

Embodiment 5. The nucleic acid compound of any one of Embodiments 1-4, wherein the 5′ segment comprises 2-5 linked nucleosides.

Embodiment 6. The nucleic acid compound of any one of Embodiments 1-5, wherein at least one nucleoside of the 5′ segment and at least one nucleoside of the 3′ segment comprises a modified sugar.

Embodiment 7. The nucleic acid compound of any one of Embodiments 1-6, wherein each nucleoside of the 5′ segment and each nucleoside of the 3′ segment comprises a modified sugar.

Embodiment 8. The nucleic acid compound of any one of Embodiments 6 or 7, wherein the modified sugar comprises a bicyclic sugar.

Embodiment 9. The nucleic acid compound of Embodiment 8, wherein the bicyclic sugar is selected from the group consisting of: 2′-O(CH2)2OCH3 (MOE); 4′-(CH2) O-2′ (LNA); 4′-(CH2)2-O-2′ (ENA); and 4′-CH(CH3)-O-2′ (cEt).

Embodiment 10. The nucleic acid compound of any one of Embodiments 1-9, wherein each internucleoside linkage is a phosphorothioate internucleoside linkage.

Embodiment 11. The nucleic acid compound of any one of Embodiments 1-10, comprising a 5-methylcytosine nucleobase in place of a non-5-methyl cytosine residue.

Embodiment 12. The nucleic acid compound of any one of Embodiments 1-11, comprising a 3-10-3 LNA-DNA-LNA gapmer, wherein all internucleoside linkages are phosphorothioate internucleoside linkages.

Embodiment 13. The nucleic acid compound of any one of Embodiments 1-11, comprising a 3-11-3 LNA-DNA-LNA gapmer, wherein all internucleoside linkages are phosphorothioate internucleoside linkages.

Embodiment 14. The nucleic acid compound of any one of Embodiments 1-11, comprising a 3-12-3 LNA-DNA-LNA gapmer, wherein all internucleoside linkages are phosphorothioate internucleoside linkages.

Embodiment 15. The nucleic acid compound of any one of Embodiments 1-11, comprising a 4-11-5 MOE-DNA-MOE gapmer, wherein all internucleoside linkages are phosphorothioate internucleoside linkages.

Embodiment 16. The nucleic acid compound of any one of Embodiments 1-15, wherein the nucleic acid compound comprises a nucleotide sequence according to SEQ ID NO: 295, 307, 309, 310, 327, 328, 329, 330, 340, 341, 342, 343, 351, 356, 359, 366, 394, 402, 523, 527, 528, 529, 539, 542, 549, 550, 551, or 553.

Embodiment 17. The nucleic acid compound of Embodiment 1, wherein the nucleic acid compound is a gapmer according to Compound ID No. 16-63326LNA, 17-29823LNA, 17-29855LNA, 17-29858LNA, 17-63264LNA, 17-63289LNA, 17-63290LNA, 17-63291LNA, 17-63324LNA, 17-63325LNA, 17-63326LNA, 17-63327LNA, 17-63458LNA, 17-67260LNA, 18-435LNA, 18-29854LNA, 18-63323LNA, 18-63458LNA, 17-63278LNA, 17-63286LNA, 17-63287LNA, 17-63288LNA, 17-63437LNA, 17-29853LNA, 18-63282LNA, 18-63285LNA, 18-63286LNA, or 18-63325LNA.

Embodiment 18. An antisense oligonucleotide (ASO) comprising: a 5′ segment of 3 linked locked nucleic acid (LNA) nucleosides, a gap segment of linked deoxynucleosides, and a 3′ segment of 3 linked LNA nucleosides, wherein all internucleoside linkages are phosphorothioate internucleoside linkages, and wherein ASO comprises the nucleotide sequence of SEQ ID NO: 550.

Embodiment 19. An antisense oligonucleotide (ASO) comprising: a 5′ segment of 3 linked locked nucleic acid (LNA) nucleosides, a gap segment of linked deoxynucleosides, and a 3′ segment of 3 linked LNA nucleosides, wherein all internucleoside linkages are phosphorothioate internucleoside linkages, and wherein ASO comprises the nucleotide sequence of SEQ ID NO: 542.

Embodiment 20. An antisense oligonucleotide (ASO) comprising: a 5′ segment of 3 linked locked nucleic acid (LNA) nucleosides, a gap segment of linked deoxynucleosides, and a 3′ segment of 3 linked LNA nucleosides, wherein all internucleoside linkages are phosphorothioate internucleoside linkages, and wherein ASO comprises the nucleotide sequence of SEQ ID NO: 394.

Embodiment 21. An antisense oligonucleotide (ASO) comprising: a 5′ segment of 3 linked locked nucleic acid (LNA) nucleosides, a gap segment of linked deoxynucleosides, and a 3′ segment of 3 linked LNA nucleosides, wherein all internucleoside linkages are phosphorothioate internucleoside linkages, and wherein ASO comprises the nucleotide sequence of SEQ ID NO: 328.

Embodiment 22. An antisense oligonucleotide (ASO) comprising: a 5′ segment of 3 linked locked nucleic acid (LNA) nucleosides, a gap segment of linked deoxynucleosides, and a 3′ segment of 3 linked LNA nucleosides, wherein all internucleoside linkages are phosphorothioate internucleoside linkages, and wherein ASO comprises the nucleotide sequence of SEQ ID NO: 342.

Embodiment 23. An antisense oligonucleotide (ASO) comprising: a 5′ segment of 3 linked locked nucleic acid (LNA) nucleosides, a gap segment of linked deoxynucleosides, and a 3′ segment of 3 linked LNA nucleosides, wherein all internucleoside linkages are phosphorothioate internucleoside linkages, and wherein ASO comprises the nucleotide sequence of SEQ ID NO: 356.

Embodiment 24. An antisense oligonucleotide (ASO) comprising: a 5′ segment of 3 linked locked nucleic acid (LNA) nucleosides, a gap segment of linked deoxynucleosides, and a 3′ segment of 3 linked LNA nucleosides, wherein all internucleoside linkages are phosphorothioate internucleoside linkages, and wherein ASO comprises the nucleotide sequence of SEQ ID NO: 295.

Embodiment 25. An antisense oligonucleotide (ASO) comprising: a 5′ segment of 3 linked locked nucleic acid (LNA) nucleosides, a gap segment of linked deoxynucleosides, and a 3′ segment of 3 linked LNA nucleosides, wherein all internucleoside linkages are phosphorothioate internucleoside linkages, and wherein ASO comprises the nucleotide sequence of SEQ ID NO: 351.

Embodiment 26. An antisense oligonucleotide (ASO) comprising: a 5′ segment of 3 linked locked nucleic acid (LNA) nucleosides, a gap segment of linked deoxynucleosides, and a 3′ segment of 3 linked LNA nucleosides, wherein all internucleoside linkages are phosphorothioate internucleoside linkages, and wherein ASO comprises the nucleotide sequence of SEQ ID NO: 307.

Embodiment 27. The nucleic acid compound or ASO of any one of Embodiments 1-26, wherein all cytosine nucleosides are replaced with 5-methylcytosine nucleosides.

Embodiment 28. The nucleic acid compound or ASO of any one of Embodiments 1-27, comprising a 2′OMe-modified nucleoside at position 2 in the DNA gap.

Embodiment 29. A composition comprising the nucleic acid compound or ASO of any one of Embodiments 1-28 or salt thereof and a pharmaceutically acceptable carrier.

Embodiment 30. The composition of Embodiment 29, wherein the composition is a pharmaceutical formulation.

Embodiment 31. A method of reducing the level of a UBE3A RNA in a cell, comprising contacting the cell with the nucleic acid compound or ASO of any one of Embodiments 1-28 or the composition of Embodiment 29 or 30, thereby reducing the level of the UBE3A RNA in the cell.

Embodiment 32. A method of inhibiting expression of UBE3A protein in a cell, comprising contacting the cell with the nucleic acid compound or ASO of any one of Embodiments 1-28 or the composition of Embodiment 29 or 30, thereby reducing expression of UBE3A protein in the cell.

Embodiment 33. The method of Embodiment 31 or 32, wherein the cell is a central nervous system neuron.

Embodiment 34. The method of any one of Embodiments 31-33, wherein the cell is triploid or tetraploid for the UBE3A gene.

Embodiment 35. The method of any one of Embodiments 31-34, wherein the cell comprises a maternal duplication of the UBE3A allele.

Embodiment 36. The method of any one of Embodiments 31-35, wherein the cell is in vitro.

Embodiment 37. The method of any one of Embodiments 31-35, wherein the cell is in a subject.

Embodiment 38. The method of Embodiment 37, wherein the subject is a human and the nucleic acid compound, ASO, or composition is administered to the subject.

Embodiment 39. The method of Embodiment 38, wherein the nucleic acid compound or ASO is administered to the subject via intravenous, subcutaneous, intramuscular, intraperitoneal, intrathecal, intra cisterna magna, or intraventricular (intracerebroventricular) injection.

Embodiment 40. A method of treating, preventing, or ameliorating a disease associated with overexpression of UBE3A in central nervous system neurons in a subject, comprising administering to the subject an nucleic acid compound or ASO of any one of Embodiments 1-28 or the composition of Embodiment 29 or 30, thereby treating, preventing, or ameliorating the disease.

Embodiment 41. The method of Embodiment 40, wherein the disease is Dup15q syndrome.

Embodiment 42. The method of Embodiment 40 or 41, wherein the subject has an idic(15) genotype.

Embodiment 43. The method of any one of Embodiments 37-42, comprising administering to the subject a second pharmaceutical agent for treating, preventing, or ameliorating one or more symptoms of Dup15q syndrome.

Embodiment 44. An nucleic acid compound or ASO according to any one of Embodiments 1-28 or the composition of Embodiment 29 or 30 for use in a method of treating, preventing, or ameliorating a disease associated with overexpression of UBE3A in central nervous system neurons in a subject.

Embodiment 45. The nucleic acid compound or ASO for use of Embodiment 44, wherein the disease is Dup15q syndrome.

Embodiment 46. The nucleic acid compound or ASO for use of Embodiment 44 or 45, wherein the subject has an idic(15) genotype.

Embodiment 47. Use of an nucleic acid compound or ASO of any one of Embodiments 1-28 or the composition of Embodiment 29 or 30 for the treatment of a disease associated with increased levels of UBE3A protein in central nervous system neurons in a subject.

Embodiment 48. Use of an nucleic acid compound or ASO of any one of Embodiments 1-28 or the composition of Embodiment 29 or 30 for the preparation of a medicament for the treatment of a disease associated with increased levels of UBE3A protein in central nervous system neurons of a subject.

Embodiment 49. The use of Embodiment 47 or 48, wherein the disease is Dup15q syndrome.

Embodiment 50. The use of any one of Embodiments 47-49, wherein the subject has an idic(15) genotype.

### Examples

The disclosure now being generally described will be more readily understood by reference to the following examples, which are included merely for purposes of illustration of certain aspects and embodiments of the present disclosure, and are not intended to limit the disclosure.

### Example 1: ASO Design and Initial Screen

Antisense oligonucleotides of 16, 17, 18, and 20 nucleotide lengths (i.e., 16-, 17-, 18- and 20-mers, respectively) were designed targeting hg38 chr15:25371667-25439024, a portion of the human UBE3A gene comprising at least a portion of the 5′ UTR through at least a portion of exon 7. The target region is provided herein as SEQ ID NO: 1. The target region was selected in part to emphasize the 5′ region of the UBE3A gene to encourage more rapid decay of mRNA transcripts and thus more potent ASOs.

In designing antisense compounds targeting the human UBE3A target region sequence (SEQ ID NO: 1), repeat regions were filtered out, 3′ guanine (G) residues were avoided, melting temperature and GC content were evaluated and taken into account, off target interactions within the human genome were avoided, and homology of target sequences among human, non-human primate (Macaca fascicularis), pig (Sus scrofa) and rodent (Mus musculus) model organisms was favored. According to these parameters, an initial set of ASOs was designed, synthesized, and evaluated in an in vitro potency screen (Table 1).

Each ASO is ascribed a unique Compound ID having the following informational components: [ASO nucleotide length]-[start position][optional SNP alteration][chemical modification]. The [ASO nucleotide length] component of each Compound ID represents the length in linked nucleosides of the ASO (e.g., “16”, “17”, “18”, or “20”). The [start position] component represents the nucleotide position of the first 5′ residue targeted by the ASO relative to SEQ ID NO: 1 (e.g., “480” or “63462”, etc.). The [optional SNP alteration] component represents certain ASOs having single nucleotide mismatches introduced relative to the target UBE3A sequence to elucidate potential impacts of single nucleotide polymorphisms (SNPs) falling within target sequences in individuals, where a single nucleotide within the ASO, either in the middle portion (SNPM) or near the 3′ end of the ASO (SNPE), was altered according to the underlined residues provided for SNPM and SNPE ASOs in Table 1. Last, the [chemical modification] component represents whether each ASO is locked nucleic acid modified or 2′-0-methoxy-ethyl modified, as further described below.

The initial set of ASOs screened included locked nucleic acid (LNA-modified) and 2′-O-methoxy-ethyl (MOE-modified) molecules. LNA-modified ASOs comprised 3 linked 5′ LNA-modified nucleosides, followed by 10-12 linked DNA nucleosides, followed by 3 linked 3′ LNA-modified nucleosides, with fully phosphorothioate-modified internucleoside linkages, i.e., 3-10-3 and 3-12-3 LNA-DNA-LNA gapmers. MOE-modified ASOs comprised 4 linked 5′ MOE-modified nucleosides followed by 11 linked DNA nucleosides, followed by 5 linked 3′ MOE-modified nucleosides, with mixed phosphorothioate-modified and phosphodiester internucleoside linkages, i.e., 4-11-5 MOE-DNA-MOE gapmers. All ASOs synthesized and screened had 5-methylcytosine in place of all cytosine residues.

Certain ASO sequences were used as positive and negative controls throughout the studies described herein. Scrambled ASOs 17-SCRA1LNA (GATTACAGATTACACAT, SEQ ID NO: 563), 18-SCRA2LNA (CATCATCATCATCATCAT, SEQ ID NO: 564), and 20-SCRA3MOE (ACATCATCATCATCATCATC, SEQ ID NO: 565) were used as negative controls. As positive controls, 20-CTRL1MOE (GGATTCAACTGCTGTCCTTG, SEQ ID NO: 566) and 20-CTRL2MOE (TGAGCTATCACCTATCCTTG, SEQ ID NO: 567) were used. Positive control ASO 20-CTRL1MOE was described in United States Pat. App. Pub. No. US/2022/0259601A1 as ASO-015 (SEQ ID NO: 298 therein) and 20-CTRL2MOE was described in Elamin et al., Stem Cell Reports 18.4 (2023): 884-898 (see Supplemental Information therein).

Briefly, mixed population human normal H9 neurons were differentiated in house from commercially available neural progenitor H9 line (Millipore-Sigma, cat #SCR055, ENStem Human Neural Progenitor Expansion Kit). A master cell bank of cryopreserved differentiated H9 neurons was generated and served as a source of neurons used in ASO selection experiments. H9 neurons were thawed and plated at 120,000 cells per well on 96 well plates, then cultured for 2 weeks to establish mature neuronal cultures. Neurons were treated with ASOs at 3 μM final concentration by gymnosis and cultured for 6 or 10 days without medium change, after which medium was removed and cells were lysed. Levels of UBE3A mRNA were evaluated using QuantiGene assay (Thermo Fisher Scientific), a hybridization-based assay for direct measurement of RNA transcripts. Both singleplex and multiplex assays were used to identify a panel of selected ASOs with consistently potent knockdown results across experiments (Table 2).

Table 2 provides UBE3A knockdown efficiency of top-performing ASOs identified by the initial singleplex and multiplex potency screens presented as a percentage of UBE3A mRNA (“% KD”) measured relative to untreated control neurons (i.e., “−50%” means a 50% reduction relative to untreated controls and “50%” means a 50% increase relative to untreated controls, as an example). Calculated standard deviation (“St Dev”) and coefficient of variation (“% CV”) are also reported for each ASO in Table 2.

TABLE 1
Human UBE3A ASOs-Initial Screen

| | | SEQ | | | SEQ |
|---|---|---|---|---|---|
| | | ID | | | ID |
| Compound ID | Sequence | NO | Compound ID | Sequence | NO |
| 16-480LNA | TGGTCGTAG | 285 | 18-63462LNA | GGTAAGCTGA | 404 |
| | TCGCCCT | | | GCTTGCTC | |
| 16-482LNA | ACTGGTCGT | 286 | 18-63463LNA | AGGTAAGCTG | 405 |
| | AGTCGCC | | | AGCTTGCT | |
| 16-29845LNA | CCAGTCTAG | 287 | 18-64667LNA | CCCCCGCAGC | 406 |
| | CTGCTAC | | | TGGCTGCA | |
| 16-29896LNA | ACATCAGGG | 288 | 18-64668LNA | TCCCCCGCAG | 407 |
| | TGATCAC | | | CTGGCTGC | |
| 16-29907LNA | GCCATTCGG | 289 | 20-431MOE | GTTCTCTTTC | 408 |
| | TGACATC | | | GCTGCCGCCA | |
| 16-29916LNA | CAAGCTGTG | 290 | 20-433MOE | TCGTTCTCTTT | 409 |
| | GCCATTC | | | CGCTGCCGC | |
| 16-29917LNA | ACAAGCTGT | 291 | 20-434MOE | CTCGTTCTCTT | 410 |
| | GGCCATT | | | TCGCTGCCG | |
| 16-31774LNA | GCAAAACAA | 292 | 20-436MOE | GCCTCGTTCT | 411 |
| | AAAATAA | | | CTTTCGCTGC | |
| 16-31920LNA | TAAGTACCC | 293 | 20-437MOE | CGCCTCGTTC | 412 |
| | CAAGATT | | | TCTTTCGCTG | |
| 16-63309LNA | CCACAGCCC | 294 | 20-439MOE | ACCGCCTCGT | 413 |
| | TCAGTTA | | | TCTCTTTCGC | |
| 16-63326LNA | TCGTGCAGG | 295 | 20-440MOE | CACCGCCTCG | 414 |
| | CTTCATT | | | TTCTCTTTCG | |
| 17-435LNA | CGTTCTCTTT | 296 | 20-441MOE | CCACCGCCTC | 415 |
| | CGCTGCC | | | GTTCTCTTTC | |
| 17-442LNA | ACCGCCTCG | 297 | 20-442MOE | GCCACCGCCT | 416 |
| | TTCTCTTT | | | CGTTCTCTTT | |
| 17-479LNA | TGGTCGTAG | 298 | 20-480MOE | TCACTGGTCG | 417 |
| | TCGCCCTC | | | TAGTCGCCCT | |
| 17-480LNA | CTGGTCGTA | 299 | 20-27066MOE | ATTCTAAAAT | 418 |
| | GTCGCCCT | | | CAGGCTCTTA | |
| 17-481LNA | ACTGGTCGT | 300 | 20-27073MOE | CTGGTGAATT | 419 |
| | AGTCGCCC | | | CTAAAATCAG | |
| 17-482LNA | CACTGGTCG | 301 | 20-27084MOE | CTTCTGAGGA | 420 |
| | TAGTCGCC | | | GCTGGTGAAT | |
| 17-27055LNA | CTCTTACAG | 302 | 20-27085MOE | ACTTCTGAGG | 421 |
| | ATTTTTAA | | | AGCTGGTGAA | |
| 17-27064LNA | AAAATCAGG | 303 | 20-27086MOE | AACTTCTGAG | 422 |
| | CTCTTACA | | | GAGCTGGTGA | |
| 17-27069LNA | ATTCTAAAA | 304 | 20-27087MOE | AAACTTCTGA | 423 |
| | TCAGGCTC | | | GGAGCTGGTG | |
| 17-27100LNA | ATATTTCGC | 305 | 20-27088MOE | CAAACTTCTG | 424 |
| | CAAACTTC | | | AGGAGCTGGT | |
| 17-29817LNA | CGTAGGCTT | 306 | 20-27090MOE | GCCAAACTTC | 425 |
| | AATAACTC | | | TGAGGAGCTG | |
| 17-29823LNA | TCTGAGCGT | 307 | 20-27091MOE | CGCCAAACTT | 426 |
| | AGGCTTAA | | | CTGAGGAGCT | |
| 17-29839LNA | CTAGCTGCT | 308 | 20-27092MOE | TCGCCAAACT | 427 |
| | ACCTTGAT | | | TCTGAGGAGC | |
| 17-29855LNA | AGGTTGTCA | 309 | 20-27093MOE | TTCGCCAAAC | 428 |
| | CACCAGTC | | | TTCTGAGGAG | |
| 17-29858LNA | AACAGGTTG | 310 | 20-29830MOE | GCTACCTTGA | 429 |
| | TCACACCA | | | TCTGAGCGTA | |
| 17-29859LNA | AAACAGGTT | 311 | 20-29831MOE | TGCTACCTTG | 430 |
| | GTCACACC | | | ATCTGAGCGT | |
| 17-29879LNA | AGCTTTGAG | 312 | 20-29836MOE | CTAGCTGCTA | 431 |
| | TCACTGAT | | | CCTTGATCTG | |
| 17-29906LNA | GCCATTCGG | 313 | 20-29838MOE | GTCTAGCTGC | 432 |
| | TGACATCA | | | TACCTTGATC | |
| 17-29907LNA | GGCCATTCG | 314 | 20-29840MOE | CAGTCTAGCT | 433 |
| | GTGACATC | | | GCTACCTTGA | |
| 17-29916LNA | ACAAGCTGT | 315 | 20-29841MOE | CCAGTCTAGC | 434 |
| | GGCCATTC | | | TGCTACCTTG | |
| 17-29917LNA | TACAAGCTG | 316 | 20-29846MOE | TCACACCAGT | 435 |
| | TGGCCATT | | | CTAGCTGCTA | |
| 17-29918LNA | TTACAAGCT | 317 | 20-29847MOE | GTCACACCAG | 436 |
| | GTGGCCAT | | | TCTAGCTGCT | |
| 17-29919LNA | TTTACAAGC | 318 | 20-29852MOE | AGGTTGTCAC | 437 |
| | TGTGGCCA | | | ACCAGTCTAG | |
| 17-29920LNA | TTTTACAAG | 319 | 20-29853MOE | CAGGTTGTCA | 438 |
| | CTGTGGCC | | | CACCAGTCTA | |
| 17-33529LNA | CGTCAGACT | 320 | 20-29854MOE | ACAGGTTGTC | 439 |
| | GAGGTTCT | | | ACACCAGTCT | |
| 17-33531LNA | GTCGTCAGA | 321 | 20-29885MOE | GGTGATCACA | 440 |
| | CTGAGGTT | | | GCTTTGAGTC | |
| 17-33541LNA | TAGCTTCAA | 322 | 20-29904MOE | GGCCATTCGG | 441 |
| | TGTCGTCA | | | TGACATCAGG | |
| 17-33545LNA | CGGCTAGCT | 323 | 20-29905MOE | TGGCCATTCG | 442 |
| | TCAATGTC | | | GTGACATCAG | |
| 17-33548LNA | ATTCGGCTA | 324 | 20-29906MOE | GTGGCCATTC | 443 |
| | GCTTCAAT | | | GGTGACATCA | |
| 17-33549LNA | CATTCGGCT | 325 | 20-29907MOE | TGTGGCCATT | 444 |
| | AGCTTCAA | | | CGGTGACATC | |
| 17-63262LNA | TTGCAGCTG | 326 | 20-29909MOE | GCTGTGGCCA | 445 |
| | CTCGCTTC | | | TTCGGTGACA | |
| 17-63264LNA | CTTTGCAGC | 327 | 20-29910MOE | AGCTGTGGCC | 446 |
| | TGCTCGCT | | | ATTCGGTGAC | |
| 17-63289LNA | GGTGGTAGT | 328 | 20-29911MOE | AAGCTGTGGC | 447 |
| | AGCGTTCT | | | CATTCGGTGA | |
| 17-63290LNA | TGGTGGTAG | 329 | 20-29912MOE | CAAGCTGTGG | 448 |
| | TAGCGTTC | | | CCATTCGGTG | |
| 17-63291LNA | CTGGTGGTA | 330 | 20-29915MOE | TTACAAGCTG | 449 |
| | GTAGCGTT | | | TGGCCATTCG | |
| 17-63306LNA | ACAGCCCTC | 331 | 20-33523MOE | CAGACTGAGG | 450 |
| | AGTTAACT | | | TTCTCCTGAT | |
| 17- | ACAGCCCTC | 332 | 20-33524MOE | TCAGACTGAG | 451 |
| 63306SNPELNA | AGTTAACA | | | GTTCTCCTGA | |
| (ACAGCCCT | | | | | |
| CAGTTAACT | | | | | |
| (SEQ ID NO: | | | | | |
| 331)): T to A | | | | | |
| 17- | ACAGCCCTA | 333 | 20-33525MOE | GTCAGACTGA | 452 |
| 63306SNPML | AGTTAACT | | | GGTTCTCCTG | |
| NA | | | | | |
| (ACAGCCCT | | | | | |
| CAGTTAACT | | | | | |
| (SEQ ID NO: | | | | | |
| 331)): C to A | | | | | |
| 17-63307LNA | CACAGCCCT | 334 | 20-33526MOE | CGTCAGACTG | 453 |
| | CAGTTAAC | | | AGGTTCTCCT | |
| 17-63308LNA | CCACAGCCC | 335 | 20-33528MOE | GTCGTCAGAC | 454 |
| | TCAGTTAA | | | TGAGGTTCTC | |
| 17-63309LNA | TCCACAGCC | 336 | 20-33529MOE | TGTCGTCAGA | 455 |
| | CTCAGTTA | | | CTGAGGTTCT | |
| 17-63320LNA | CAGGCTTCA | 337 | 20-33530MOE | ATGTCGTCAG | 456 |
| | TTTCCACA | | | ACTGAGGTTC | |
| 17-63321LNA | GCAGGCTTC | 338 | 20-33532MOE | CAATGTCGTC | 457 |
| | ATTTCCAC | | | AGACTGAGGT | |
| 17-63323LNA | GTGCAGGCT | 339 | 20-33536MOE | GCTTCAATGT | 458 |
| | TCATTTCC | | | CGTCAGACTG | |
| 17-63324LNA | CGTGCAGGC | 340 | 20-33540MOE | GCTAGCTTCA | 459 |
| | TTCATTTC | | | ATGTCGTCAG | |
| 17-63325LNA | TCGTGCAGG | 341 | 20-33541MOE | GGCTAGCTTC | 460 |
| | CTTCATTT | | | AATGTCGTCA | |
| 17-63326LNA | TTCGTGCAG | 342 | 20-33542MOE | CGGCTAGCTT | 461 |
| | GCTTCATT | | | CAATGTCGTC | |
| 17-63327LNA | ATTCGTGCA | 343 | 20-33543MOE | TCGGCTAGCT | 462 |
| | GGCTTCAT | | | TCAATGTCGT | |
| 17-63340LNA | AAGCACAAA | 344 | 20-33544MOE | TTCGGCTAGC | 463 |
| | ACTCATTC | | | TTCAATGTCG | |
| 17-63348LNA | TGGACAGGA | 345 | 20-63263MOE | TGCTTTGCAG | 464 |
| | AGCACAAA | | | CTGCTCGCTT | |
| 17-63351LNA | AGTTGGACA | 346 | 20-63264MOE | ATGCTTTGCA | 465 |
| | GGAAGCAC | | | GCTGCTCGCT | |
| 17-63352LNA | AAGTTGGAC | 347 | 20-63265MOE | GATGCTTTGC | 466 |
| | AGGAAGCA | | | AGCTGCTCGC | |
| 17-63364LNA | CCATACGAA | 348 | 20-63266MOE | AGATGCTTTG | 467 |
| | GAAAAGTT | | | CAGCTGCTCG | |
| 17-63410LNA | TTAATCTTA | 349 | 20-63288MOE | CTGGTGGTAG | 468 |
| | TAAAGCTC | | | TAGCGTTCTA | |
| 17-63457LNA | CTGAGCTTG | 350 | 20-63289MOE | ACTGGTGGTA | 469 |
| | CTCCTTTC | | | GTAGCGTTCT | |
| 17-63458LNA | GCTGAGCTT | 351 | 20-63290MOE | AACTGGTGGT | 470 |
| | GCTCCTTT | | | AGTAGCGTTC | |
| 17-63463LNA | GGTAAGCTG | 352 | 20-63293MOE | GTTAACTGGT | 471 |
| | AGCTTGCT | | | GGTAGTAGCG | |
| 17-63467LNA | TCAAGGTAA | 353 | 20-63301MOE | AGCCCTCAGT | 472 |
| | GCTGAGCT | | | TAACTGGTGG | |
| 17-67212LNA | GTTAAGTAA | 354 | 20-63302MOE | CAGCCCTCAG | 473 |
| | GTCACATC | | | TTAACTGGTG | |
| 17-67242LNA | AATTCAAGA | 355 | 20-63303MOE | ACAGCCCTCA | 474 |
| | ATTTCATA | | | GTTAACTGGT | |
| 17-67260LNA | TCCTCTCTTT | 356 | 20-63304MOE | CACAGCCCTC | 475 |
| | CTCTACA | | | AGTTAACTGG | |
| 17-67307LNA | CAGCACTAG | 357 | 20-63305MOE | CCACAGCCCT | 476 |
| | AAAAAACT | | | CAGTTAACTG | |
| 17-67312LNA | TGCCTCAGC | 358 | 20-63306MOE | TCCACAGCCC | 477 |
| | ACTAGAAA | | | TCAGTTAACT | |
| 18-435LNA | TCGTTCTCTT | 359 | 20-63307MOE | TTCCACAGCC | 478 |
| | TCGCTGCC | | | CTCAGTTAAC | |
| 18-438LNA | GCCTCGTTC | 360 | 20- | TTCCACAGCC | 479 |
| | TCTTTCGCT | | 63307SNPEMOE | CTCAGTTGAC | |
| | | | (TTCCACAGC | | |
| | | | CCTCAGTTAA | | |
| | | | C (SEQ ID NO: | | |
| | | | 478)): A to G | | |
| 18-480LNA | ACTGGTCGT | 361 | 20- | TTCCACAGCG | 480 |
| | AGTCGCCCT | | 63307SNPMMOE | CTCAGTTAAC | |
| | | | (TTCCACAGC | | |
| | | | CCTCAGTTAA | | |
| | | | C (SEQ ID NO: | | |
| | | | 478)): C to G | | |
| 18-482LNA | TCACTGGTC | 362 | 20-63310MOE | CATTTCCACA | 481 |
| | GTAGTCGCC | | | GCCCTCAGTT | |
| 18-861LNA | CCTGTCTGG | 363 | 20-63311MOE | TCATTTCCAC | 482 |
| | GGCCCCGGC | | | AGCCCTCAGT | |
| 18-27091LNA | CCAAACTTC | 364 | 20-63317MOE | CAGGCTTCAT | 483 |
| | TGAGGAGCT | | | TTCCACAGCC | |
| 18-29838LNA | CTAGCTGCT | 365 | 20-63318MOE | GCAGGCTTCA | 484 |
| | ACCTTGATC | | | TTTCCACAGC | |
| 18-29854LNA | AGGTTGTCA | 366 | 20-63319MOE | TGCAGGCTTC | 485 |
| | CACCAGTCT | | | ATTTCCACAG | |
| 18-29906LNA | GGCCATTCG | 367 | 20-63320MOE | GTGCAGGCTT | 486 |
| | GTGACATCA | | | CATTTCCACA | |
| 18-29907LNA | TGGCCATTC | 368 | 20-63321MOE | CGTGCAGGCT | 487 |
| | GGTGACATC | | | TCATTTCCAC | |
| 18-29916LNA | TACAAGCTG | 369 | 20-63322MOE | TCGTGCAGGC | 488 |
| | TGGCCATTC | | | TTCATTTCCA | |
| 18-29920LNA | CTTTTACAA | 370 | 20-63323MOE | TTCGTGCAGG | 489 |
| | GCTGTGGCC | | | CTTCATTTCC | |
| 18-33523LNA | GACTGAGGT | 371 | 20-63327MOE | CTCATTCGTG | 490 |
| | TCTCCTGAT | | | CAGGCTTCAT | |
| 18-33526LNA | TCAGACTGA | 372 | 20-63332MOE | CAAAACTCAT | 491 |
| | GGTTCTCCT | | | TCGTGCAGGC | |
| 18- | TCAGACTGA | 373 | 20-63344MOE | GGACAGGAA | 492 |
| 33526SNPELNA | GGTTCTCCC | | | GCACAAAACT | |
| (TCAGACTG | | | | | |
| AGGTTCTCC | | | | | |
| T (SEQ ID | | | | | |
| NO: 372)): | | | | | |
| T to C | | | | | |
| 18- | TCAGACTGA | 374 | 20-63411MOE | TGCATTAATC | 493 |
| 33526SNPMLNA | CGTTCTCCT | | | TTATAAAGCT | |
| (TCAGACTG | | | | | |
| AGGTTCTCC | | | | | |
| T (SEQ ID | | | | | |
| NO: 372)): | | | | | |
| G to C | | | | | |
| 18-33528LNA | CGTCAGACT | 375 | 20-63450MOE | GCTTGCTCCT | 494 |
| | GAGGTTCTC | | | TTCTTGGAGG | |
| 18-33530LNA | GTCGTCAGA | 376 | 20-63451MOE | AGCTTGCTCC | 495 |
| | CTGAGGTTC | | | TTTCTTGGAG | |
| 18-33531LNA | TGTCGTCAG | 377 | 20-63452MOE | GAGCTTGCTC | 496 |
| | ACTGAGGTT | | | CTTTCTTGGA | |
| 18-33538LNA | GCTTCAATG | 378 | 20-63453MOE | TGAGCTTGCT | 497 |
| | TCGTCAGAC | | | CCTTTCTTGG | |
| 18-33542LNA | GCTAGCTTC | 379 | 20-63454MOE | CTGAGCTTGC | 498 |
| | AATGTCGTC | | | TCCTTTCTTG | |
| 18-33545LNA | TCGGCTAGC | 380 | 20-63455MOE | GCTGAGCTTG | 499 |
| | TTCAATGTC | | | CTCCTTTCTT | |
| 18-63262LNA | TTTGCAGCT | 381 | 20-63456MOE | AGCTGAGCTT | 500 |
| | GCTCGCTTC | | | GCTCCTTTCT | |
| 18-63263LNA | CTTTGCAGC | 382 | 20-63457MOE | AAGCTGAGCT | 501 |
| | TGCTCGCTT | | | TGCTCCTTTC | |
| 18-63264LNA | GCTTTGCAG | 383 | 20-63459MOE | GTAAGCTGAG | 502 |
| | CTGCTCGCT | | | CTTGCTCCTT | |
| 18-63268LNA | AGATGCTTT | 384 | 20-63460MOE | GGTAAGCTGA | 503 |
| | GCAGCTGCT | | | GCTTGCTCCT | |
| 18-63290LNA | CTGGTGGTA | 385 | 20-63461MOE | AGGTAAGCTG | 504 |
| | GTAGCGTTC | | | AGCTTGCTCC | |
| 18-63291LNA | ACTGGTGGT | 386 | 20-63462MOE | AAGGTAAGCT | 505 |
| | AGTAGCGTT | | | GAGCTTGCTC | |
| 18-63306LNA | CACAGCCCT | 387 | 20-63463MOE | CAAGGTAAGC | 506 |
| | CAGTTAACT | | | TGAGCTTGCT | |
| 18-63307LNA | CCACAGCCC | 388 | 20-63464MOE | TCAAGGTAAG | 507 |
| | TCAGTTAAC | | | CTGAGCTTGC | |
| 18-63308LNA | TCCACAGCC | 389 | 20-63478MOE | CACCTTTCGA | 508 |
| | CTCAGTTAA | | | GTTCTCAAGG | |
| 18-63309LNA | TTCCACAGC | 390 | 20-63479MOE | GCACCTTTCG | 509 |
| | CCTCAGTTA | | | AGTTCTCAAG | |
| 18-63320LNA | GCAGGCTTC | 391 | 20-67202MOE | AAGTCACATC | 510 |
| | ATTTCCACA | | | TAGAAAATCA | |
| 18-63321LNA | TGCAGGCTT | 392 | 20-67248MOE | TCTCTACATA | 511 |
| | CATTTCCAC | | | ATTCAAGAAT | |
| 18-63322LNA | GTGCAGGCT | 393 | 20-67296MOE | GAAAAAACTC | 512 |
| | TCATTTCCA | | | TTCCAATAAC | |
| 18-63323LNA | CGTGCAGGC | 394 | 20-67319MOE | TCTGTACCAA | 513 |
| | TTCATTTCC | | | TGCCTCAGCA | |
| 18-63324LNA | TCGTGCAGG | 395 | 20-67320MOE | CTCTGTACCA | 514 |
| | CTTCATTTC | | | ATGCCTCAGC | |
| 18-63327LNA | CATTCGTGC | 396 | 20-67321MOE | GCTCTGTACC | 515 |
| | AGGCTTCAT | | | AATGCCTCAG | |
| 18-63346LNA | GGACAGGA | 397 | 20-67323MOE | AAGCTCTGTA | 516 |
| | AGCACAAAA | | | CCAATGCCTC | |
| | C | | | | |
| 18-63350LNA | AGTTGGACA | 398 | 17-SCRAILNA | GATTACAGAT | 563 |
| | GGAAGCACA | | | TACACAT | |
| 18-63351LNA | AAGTTGGAC | 399 | 18-SCRA2LNA | CATCATCATC | 564 |
| | AGGAAGCAC | | | ATCATCAT | |
| 18-63456LNA | CTGAGCTTG | 400 | 20-SCRA3MOE | ACATCATCAT | 565 |
| | CTCCTTTCT | | | CATCATCATC | |
| 18-63457LNA | GCTGAGCTT | 401 | 20-CTRL1MOE | GGATTCAACT | 566 |
| | GCTCCTTTC | | | GCTGTCCTTG | |
| 18-63458LNA | AGCTGAGCT | 402 | 20-CTRL2MOE | TGAGCTATCA | 567 |
| | TGCTCCTTT | | | CCTATCCTTG | |
| 18-63460LNA | TAAGCTGAG | 403 | | | |
| | CTTGCTCCT | | | | |

TABLE 2
Selected ASOs from Initial Screen

| | | SEQ ID | | | |
|---|---|---|---|---|---|
| Compound ID | Sequence | NO | %KD | St Dev | %CV |
| 17-63290LNA | TGGTGGTAGTAGCGTTC | 329 | −74% | 0.002 | 7% |
| 16-63326LNA | TCGTGCAGGCTTCATT | 295 | −67% | 0.001 | 2% |
| 17-63326LNA | TTCGTGCAGGCTTCATT | 342 | −65% | 0.001 | 4% |
| 17-63291LNA | CTGGTGGTAGTAGCGTT | 330 | −65% | 0.003 | 8% |
| 17-29823LNA | TCTGAGCGTAGGCTTAA | 307 | −65% | 0.003 | 9% |
| 18-435LNA | TCGTTCTCTTTCGCTGCC | 359 | −64% | 0.001 | 4% |
| 17-63289LNA | GGTGGTAGTAGCGTTCT | 328 | −64% | 0.003 | 8% |
| 17-63458LNA | GCTGAGCTTGCTCCTTT | 351 | −63% | 0.001 | 2% |
| 17-29855LNA | AGGTTGTCACACCAGTC | 309 | −63% | 0.008 | 22% |
| 17-29858LNA | AACAGGTTGTCACACCA | 310 | −62% | 0.001 | 2% |
| 17-63324LNA | CGTGCAGGCTTCATTTC | 340 | −61% | 0.012 | 29% |
| 18-63323LNA | CGTGCAGGCTTCATTTCC | 394 | −61% | 0.001 | 4% |
| 17-63264LNA | CTTTGCAGCTGCTCGCT | 327 | −61% | 0.005 | 12% |
| 18-29854LNA | AGGTTGTCACACCAGTCT | 366 | −61% | 0.005 | 11% |
| 17-67260LNA | TCCTCTCTTTCTCTACA | 356 | −60% | 0.002 | 5% |
| 17-63325LNA | TCGTGCAGGCTTCATTT | 341 | −60% | 0.007 | 15% |
| 18-63458LNA | AGCTGAGCTTGCTCCTTT | 402 | −60% | 0.005 | 12% |
| 17-63327LNA | ATTCGTGCAGGCTTCAT | 343 | −58% | 0.009 | 19% |

Analysis of the ASOs described in Table 2 revealed that many of top-performing ASOs clustered in regions of the target UBE3A locus, suggesting these regions were highly susceptible to knockdown by antisense mechanisms. These “hot spots” were then used as target sequences for the design of additional ASOs, as described in Example 2.

### Example 2: Identification of Hot Spots & Secondary ASO Screen

The discovery of certain regions in the UBE3A target sequence justified design and screening of additional ASOs targeting these hot spots, regions where highly potent ASOs clustered within the UBE3A target sequence. As the initial screen did not include all designed ASOs but only a selection closely tiled along the UBE3A target sequence for hot spots identification, all the remaining ASOs designed for these hot spot regions were subsequently tested. The additional ASOs were screened for knockdown potency in normal human neurons. Neurons were cultured as in Example 1 and were treated with 3 μM ASO for 10 days. Results of the secondary screen of hot spot ASOs are provided in Table 3, where UBE3A knockdown efficiency is presented as a percentage of UBE3A mRNA (“% KD”) measured relative to untreated control neurons (i.e., “−50%” means a 50% reduction relative to untreated controls and “50%” means a 50% increase relative to untreated controls, as an example). Expression levels normalized to untreated controls (“Norm to UT”) are also provided.

TABLE 3
Hot Spot ASOs-Secondary Screen

| | | SEQ ID | | Norm to |
|---|---|---|---|---|
| Compound ID | Sequence | NO | %KD | UT |
| 18-63285LNA | GGTAGTAGCGTTCTATTA | 550 | −82% | 0.18 |
| 17-29853LNA | GTTGTCACACCAGTCTA | 542 | −81% | 0.19 |
| 17-29853LNA | GTTGTCACACCAGTCTA | 542 | −79% | 0.21 |
| 17-63286LNA | GGTAGTAGCGTTCTATT | 527 | −74% | 0.26 |
| 18-63286LNA | TGGTAGTAGCGTTCTATT | 551 | −71% | 0.29 |
| 18-63325LNA | TTCGTGCAGGCTTCATTT | 553 | −70% | 0.30 |
| 17-63437LNA | GAGGGATGAGGATCACA | 539 | −68% | 0.32 |
| 17-63278LNA | CGTTCTATTAGATGCTT | 523 | −67% | 0.33 |
| 17-63288LNA | GTGGTAGTAGCGTTCTA | 529 | −65% | 0.35 |
| 17-63287LNA | TGGTAGTAGCGTTCTAT | 528 | −65% | 0.35 |
| 18-63282LNA | AGTAGCGTTCTATTAGAT | 549 | −65% | 0.35 |
| 17-63278LNA | CGTTCTATTAGATGCTT | 523 | −64% | 0.36 |
| 20-432MOE | CGTTCTCTTTCGCTGCCGCC | 562 | −62% | 0.38 |
| 18-63437LNA | GGAGGGATGAGGATCACA | 555 | −62% | 0.38 |
| 18-63287LNA | GTGGTAGTAGCGTTCTAT | 552 | −62% | 0.38 |
| 17-29854LNA | GGTTGTCACACCAGTCT | 543 | −61% | 0.39 |
| 17-435LNA | CGTTCTCTTTCGCTGCC | 296 | −59% | 0.41 |
| 17-29822LNA | CTGAGCGTAGGCTTAAT | 521 | −58% | 0.42 |
| 17-63282LNA | GTAGCGTTCTATTAGAT | 525 | −57% | 0.43 |
| 20- | TGAGCTATCACCTATCCTTG | 567 | −57% | 0.43 |
| CTRL2MOE | | | | |
| 17-29817LNA | CGTAGGCTTAATAACTC | 306 | −55% | 0.45 |
| 17-63285LNA | GTAGTAGCGTTCTATTA | 526 | −55% | 0.45 |
| 18-63439LNA | TTGGAGGGATGAGGATCA | 556 | −55% | 0.45 |
| 17-63343LNA | AGGAAGCACAAAACTCA | 533 | −54% | 0.46 |
| 17-63343LNA | AGGAAGCACAAAACTCA | 533 | −53% | 0.47 |
| 20- | GGATTCAACTGCTGTCCTTG | 566 | −51% | 0.49 |
| CTRL1MOE | | | | |
| 17-63441LNA | CTTGGAGGGATGAGGAT | 540 | −49% | 0.51 |
| 18-63326LNA | ATTCGTGCAGGCTTCATT | 554 | −48% | 0.52 |
| 17-63296LNA | GTTAACTGGTGGTAGTA | 530 | −48% | 0.52 |
| 18-29859LNA | AAAACAGGTTGTCACACC | 544 | −48% | 0.52 |
| 17-63299LNA | TCAGTTAACTGGTGGTA | 531 | −46% | 0.54 |
| 17-63346LNA | GACAGGAAGCACAAAAC | 534 | −44% | 0.56 |
| 18-63440LNA | CTTGGAGGGATGAGGATC | 557 | −43% | 0.57 |
| 18-29838LNA | CTAGCTGCTACCTTGATC | 365 | −43% | 0.57 |
| 17-63342LNA | GGAAGCACAAAACTCAT | 532 | −41% | 0.59 |
| 17-29821LNA | TGAGCGTAGGCTTAATA | 520 | −40% | 0.60 |
| 18-63279LNA | AGCGTTCTATTAGATGCT | 548 | −40% | 0.60 |
| 17-29847LNA | ACACCAGTCTAGCTGCT | 541 | −39% | 0.61 |
| 18-63274LNA | TCTATTAGATGCTTTGCA | 546 | −36% | 0.64 |
| 18-63278LNA | GCGTTCTATTAGATGCTT | 547 | −36% | 0.64 |
| 16-480LNA | TGGTCGTAGTCGCCCT | 285 | −36% | 0.64 |
| 20-424MOE | TTCGCTGCCGCCACCGCCG | 559 | −34% | 0.66 |
| | G | | | |
| 20-426MOE | CTTTCGCTGCCGCCACCGCC | 561 | −32% | 0.68 |
| 17-29820LNA | GAGCGTAGGCTTAATAA | 519 | −30% | 0.70 |
| 20-425MOE | TTTCGCTGCCGCCACCGCC | 560 | −26% | 0.74 |
| | G | | | |
| 17-63279LNA | GCGTTCTATTAGATGCT | 524 | −25% | 0.75 |
| 17-63274LNA | CTATTAGATGCTTTGCA | 522 | −25% | 0.75 |
| 17-63402LNA | ATAAAGCTCGAGGGCTT | 538 | −24% | 0.76 |
| 17-479LNA | TGGTCGTAGTCGCCCTC | 298 | −24% | 0.76 |
| 17-480LNA | CTGGTCGTAGTCGCCCT | 299 | −22% | 0.78 |
| 17-480LNA | CTGGTCGTAGTCGCCCT | 299 | −20% | 0.80 |
| 18-63447LNA | CTCCTTTCTTGGAGGGAT | 558 | −20% | 0.80 |
| 17-29818LNA | GCGTAGGCTTAATAACT | 517 | −20% | 0.80 |
| 17-63340LNA | AAGCACAAAACTCATTC | 344 | −19% | 0.81 |
| 17-29819LNA | AGCGTAGGCTTAATAAC | 518 | −19% | 0.81 |
| 17-63399LNA | AAGCTCGAGGGCTTTAA | 536 | −16% | 0.84 |
| 17-63398LNA | AGCTCGAGGGCTTTAAT | 535 | −14% | 0.86 |
| 18-29860LNA | AAAAACAGGTTGTCACAC | 545 | −14% | 0.86 |
| 17-63401LNA | TAAAGCTCGAGGGCTTT | 537 | −11% | 0.89 |
| 18-SCRA2LNA | CATCATCATCATCATCAT | 564 | −3% | 0.97 |

### Example 3: Replicate Study Confirms Potency of Top Performing ASOs

Results from initial and secondary ASO screens were evaluated and an augmented panel of the top 30 ASOs was selected for repeat studies to confirm knockdown potency in normal human neurons. Neurons were cultured as in Example 1 and were treated with 3 μM ASO for 10 days. Normalized UBE3A mRNA levels were determined using multiplex QuantiGene assay. Results are provided in Table 4.

TABLE 4
Replicate Potency Study Results

| | | SEQ | |
|---|---|---|---|
| Compound ID | Sequence | ID NO | %KD |
| 18-63285LNA | GGTAGTAGCGTTCTATTA | 550 | −95% |
| 17-29853LNA | GTTGTCACACCAGTCTA | 542 | −88% |
| 18-63286LNA | TGGTAGTAGCGTTCTATT | 551 | −82% |
| 18-435LNA | TCGTTCTCTTTCGCTGCC | 359 | −81% |
| 17-63286LNA | GGTAGTAGCGTTCTATT | 527 | −80% |
| 17-63324LNA | CGTGCAGGCTTCATTTC | 340 | −78% |
| 18-63323LNA | CGTGCAGGCTTCATTTCC | 394 | −77% |
| 17-63325LNA | TCGTGCAGGCTTCATTT | 341 | −76% |
| 17-63289LNA | GGTGGTAGTAGCGTTCT | 328 | −76% |
| 17-63326LNA | TTCGTGCAGGCTTCATT | 342 | −76% |
| 17-63290LNA | TGGTGGTAGTAGCGTTC | 329 | −75% |
| 17-67260LNA | TCCTCTCTTTCTCTACA | 356 | −75% |
| 17-63291LNA | CTGGTGGTAGTAGCGTT | 330 | −74% |
| 16-63326LNA | TCGTGCAGGCTTCATT | 295 | −74% |
| 17-63327LNA | ATTCGTGCAGGCTTCAT | 343 | −73% |
| 17-63458LNA | GCTGAGCTTGCTCCTTT | 351 | −73% |
| 18-63458LNA | AGCTGAGCTTGCTCCTTT | 402 | −73% |
| 17-29823LNA | TCTGAGCGTAGGCTTAA | 307 | −73% |
| 17-29855LNA | AGGTTGTCACACCAGTC | 309 | −72% |
| 18-29854LNA | AGGTTGTCACACCAGTCT | 366 | −72% |
| 17-29823LNA | TCTGAGCGTAGGCTTAA | 307 | −70% |
| 17-29855LNA | AGGTTGTCACACCAGTC | 309 | −70% |
| 18-63325LNA | TTCGTGCAGGCTTCATTT | 553 | −65% |
| 17-63287LNA | TGGTAGTAGCGTTCTAT | 528 | −64% |
| 17-63264LNA | CTTTGCAGCTGCTCGCT | 327 | −61% |
| 17-63278LNA | CGTTCTATTAGATGCTT | 523 | −59% |
| 17-63288LNA | GTGGTAGTAGCGTTCTA | 529 | −56% |
| 17-63437LNA | GAGGGATGAGGATCACA | 539 | −55% |
| 17-29858LNA | AACAGGTTGTCACACCA | 310 | −53% |
| 18-63282LNA | AGTAGCGTTCTATTAGAT | 549 | −51% |

Select ASOs from the replicated potency study shown in Table 4 were carried forward for evaluation of dose response and calculation of half-maximal inhibitory concentration (IC50).

### Example 4: Dose Response Studies in Normal Human Neurons

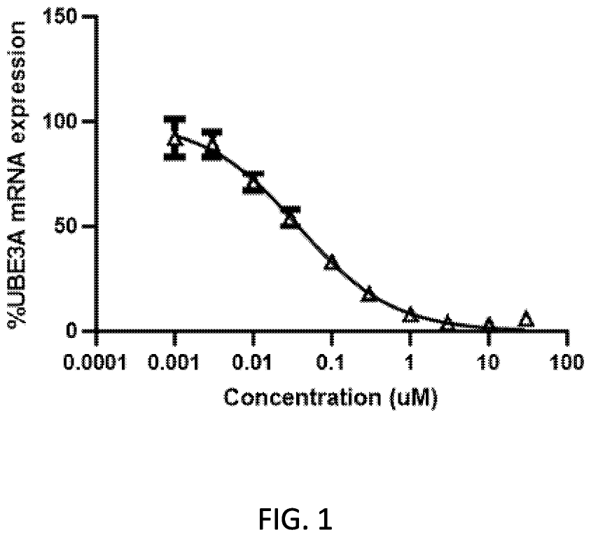

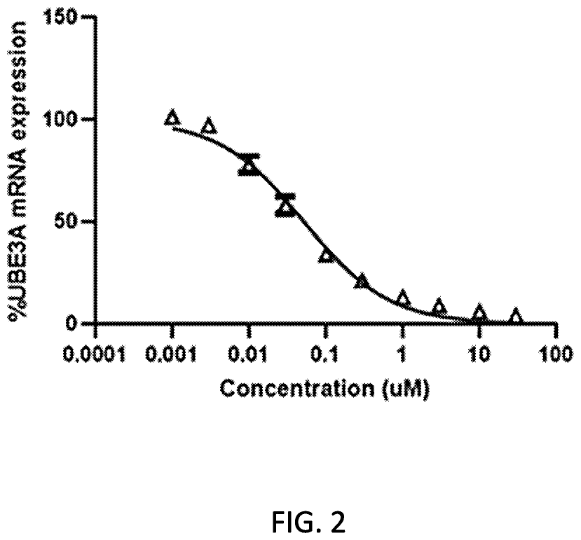

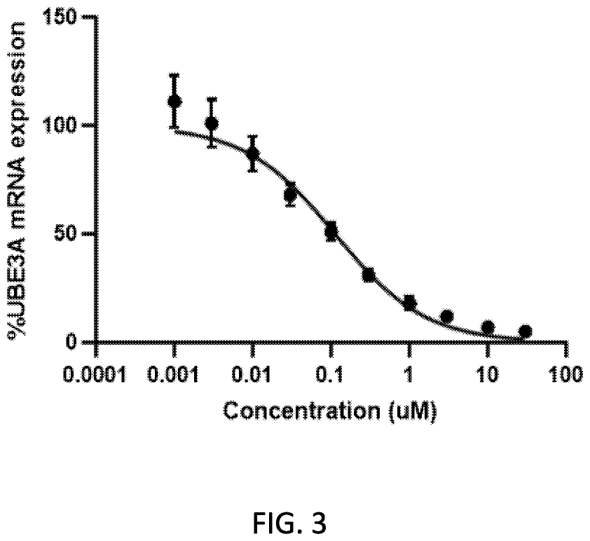

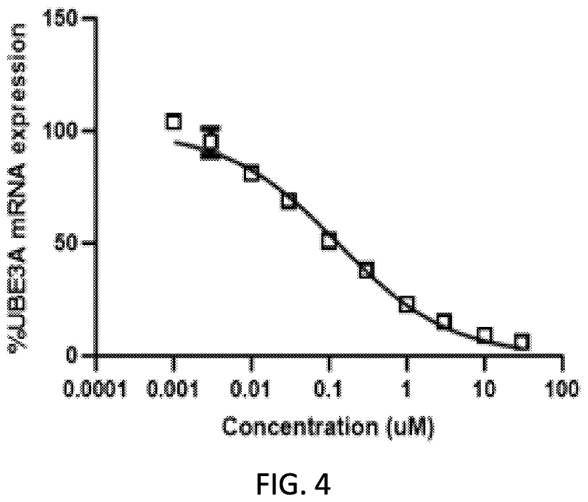

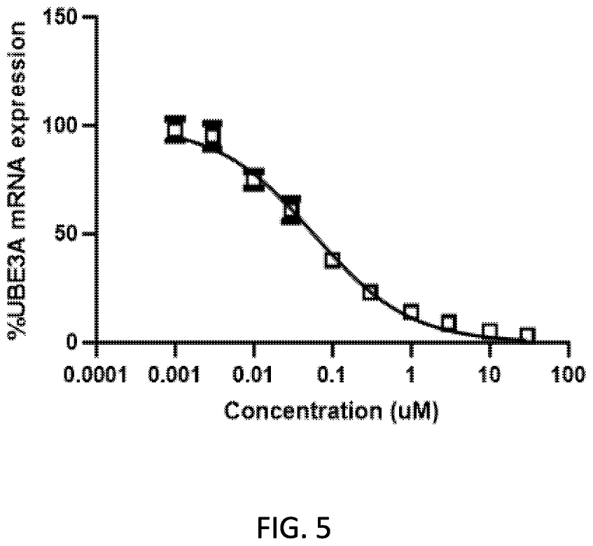

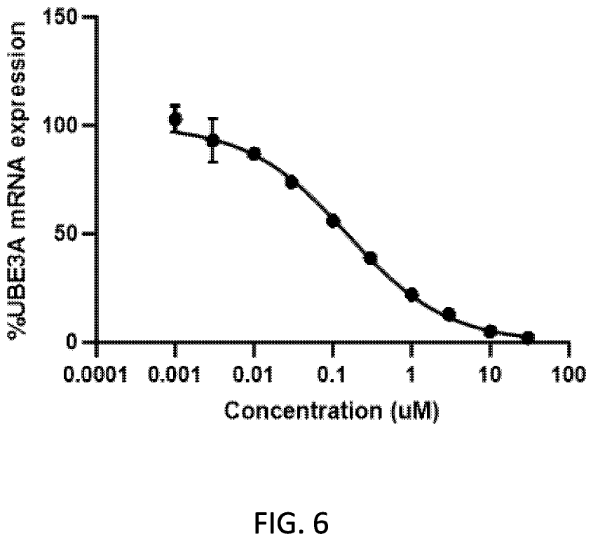

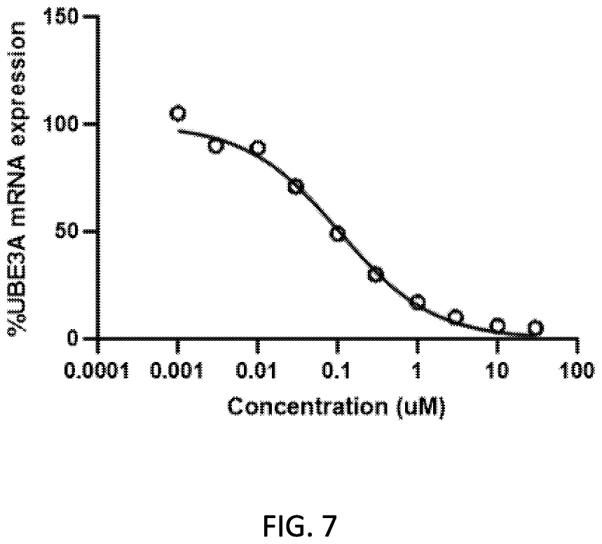

Dose response studies were carried out to determine IC50 values for select ASOs following the confirmatory potency screen of Example 3. Semi-log dilutions ranging from 30 μM to 1 nM of select ASOs were applied to normal human neurons (H9) for 10 days (Table 5). UBE3A mRNA levels were measured using multiplex QuantiGene assay. IC50 values were calculated by generation of four parameters nonlinear regression curves using GraphPad Prism software. IC50, confidence intervals (“Conf. Int.”) and R-squared (“R-sq.”) analysis are provided in Table 5. Dose response curves for each of the select ASOs are shown in FIG. 1 through FIG. 7.

TABLE 5
IC50 Analysis of Select ASOs in Human Neurons

| Compound | | SEQ ID | | Conf. | | |
|---|---|---|---|---|---|---|
| ID | Sequence | NO | IC50 | Int. | R-sq. | FIG |
| 18- | GGTAGTAGCGTTCTA | 550 | 37 | 32 to 43 | 0.9876 | 1 |
| 63285LNA | TTA | | nM | | | |
| 17- | GTTGTCACACCAGTC | 542 | 52 | 44 to 61 | 0.9855 | 2 |
| 29853LNA | TA | | nM | | | |
| 18- | CGTGCAGGCTTCATT | 394 | 112 | 84 to | 0.9613 | 3 |
| 63323LNA | TCC | | nM | 150 | | |
| 17- | TCCTCTCTTTCTCTAC | 356 | 130 | 109 to | 0.9857 | 4 |
| 67260LNA | A | | nM | 155 | | |
| 16- | TCGTGCAGGCTTCAT | 295 | 57 | 49 to 67 | 0.9873 | 5 |
| 63326LNA | T | | nM | | | |
| 17- | GCTGAGCTTGCTCCTT | 351 | 151 | 128 to | 0.9914 | 6 |
| 63458LNA | T | | nM | 179 | | |
| 17- | TCTGAGCGTAGGCTT | 307 | 105 | 91 to | 0.9721 | 7 |
| 29823LNA | AA | | nM | 121 | | |

Based on this analysis, potency of the ASOs was determined and candidates were rank-ordered according to potency.

### Example 5: Potent Reduction of Over-Expressed UBE3A RNA and Protein in Dup15q-idic Patient iPSC-Derived Neurons

Patient-derived induced pluripotent stem cells (iPSCs) from a Dup15q patient having idic(15) genotype (Dup1-8), as well as, its engineered isogenic control iPSC line (Dup 1-8-corr), in which supernumerary chromosome containing additional 2 copies of UBE3A was removed by CRISPR approach (Elamin et al, 2022), were purchased from the University of Connecticut (UCONN, Technology Commercialization Services Office of the Vice President for Research). iPSCs were differentiated into neurons by highly efficient SMAD inhibition-mediated neural induction method using STEMdiff SMADi Neural Induction Kit (Stem Cell Technologies). Relative UBE3A genomic DNA levels in iPSC lines were evaluated by qPCR and were found to be approximately 2× control (corrected) levels in Dup15q idic line as expected in view of the tetraploid idic(15) genotype compared to wild type diploid cells (data not shown). Relative UBE3A mRNA levels in differentiated neurons were evaluated by multiplex QuantiGene assay and were found to be approximately 2× control levels in Dup15q idic neurons. Due to silencing of paternal allele of UBE3A by antisense transcript (ATS) in neurons, the expected level of UBE3A mRNA in Dup15q idic compared to isogenic control neurons is 3:1. Nevertheless, the relationship of copy number to expressed levels seems not to be linear, as concluded from studies in postmortem brain tissue from Dup15q patients (Scoles et al., 2011; Molecular Autism, 2(1)).

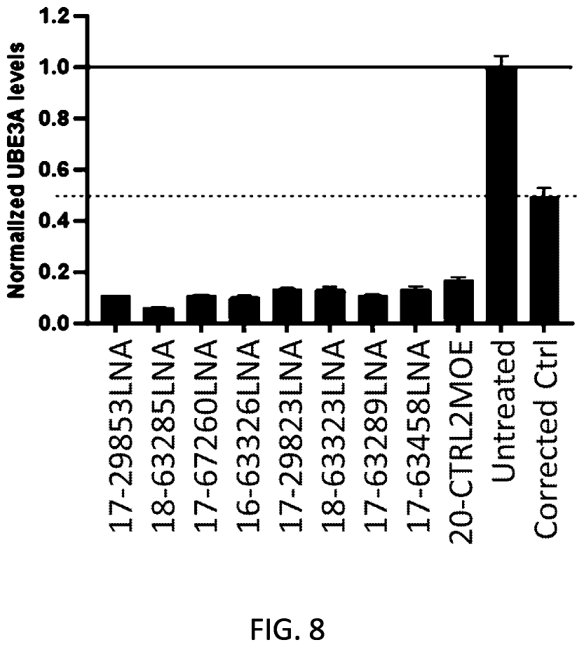

To evaluate effects on UBE3A mRNA levels, iPSC-derived Dup15q-idic(15) neurons were cultured in the presence of 3 μM ASOs for 10 days (triplicates). UBE3A mRNA was measured and compared to untreated controls. Results are shown in FIG. 8.

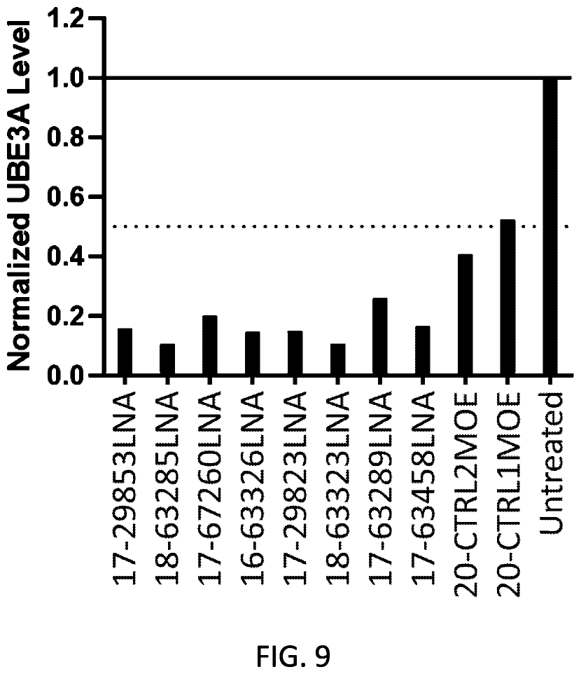

To evaluate effects on UBE3A protein levels, iPSC-derived Dup15q-idic(15) neurons were cultured in the presence of 3 μM ASOs for 10 days (triplicates). UBE3A protein was measured (from single well, N=1) and compared to untreated controls. Results are shown in FIG. 9.

In conclusion, in the context of UBE3A over-expression in human iPSC-derived Dup15q-idic(15) neurons, ASOs identified in these studies show potent reduction of UBE3A mRNA and protein levels.

### Example 6: Dose Response Studies in Dup15q-idic(15) Neurons

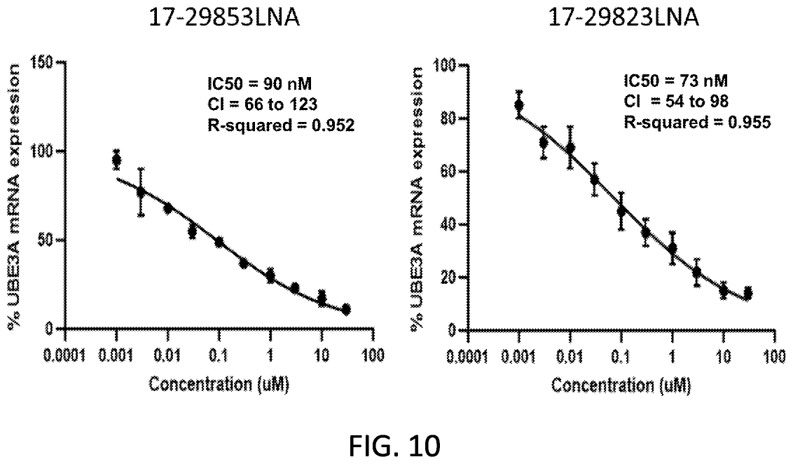

Dose response studies were carried out in Dup15q-idic(15) neurons to determine IC50 values for select ASOs (17-29853LNA and 17-29823LNA). Semi-log dilutions ranging from 30 μM to 1 nM of each ASO were applied to idic(15) neurons in culture for 10 days. Results, including IC50, confidence intervals (“CI”) and R-squared analysis are provided in FIG. 10.

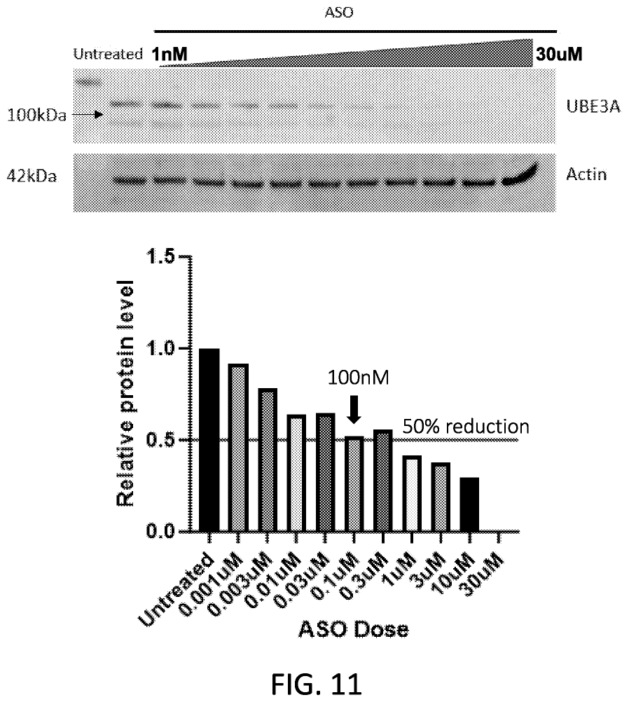

Dose-dependent UBE3A protein knockdown was also confirmed by Western blot for ASO 17-29823LNA, as shown in FIG. 11, where relative UBE3A protein levels following treatment with semi-log dilutions from 30 μM to 1 nM of 17-29823LNA are shown.

These data confirm that, despite over expression of UBE3A, dose dependent target reduction follows similar pattern in Dup15q idic neurons compared to normal human neurons (H9).

### Example 7. In Vivo Tolerability Study

12 candidate ASOs were selected for in vivo tolerability screening in mice. Of 12 ASOs tested, ASOs according to Compound ID 18-63285LNA, 17-63286LNA, and 17-63458LNA were found to have favorable tolerability profiles.

### Example 8. In Vivo Target Engagement

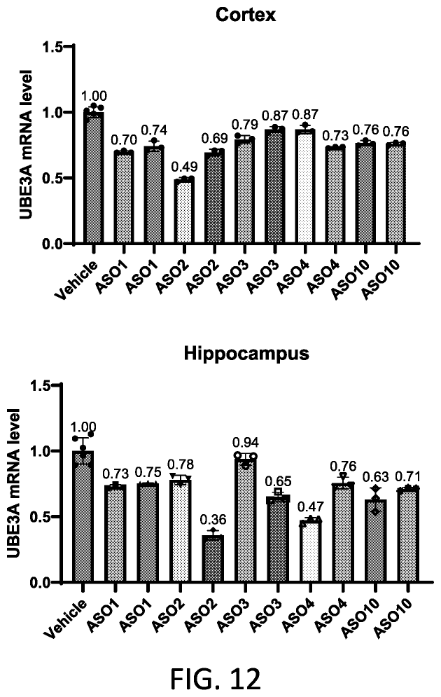

18-63285LNA (ASO1), 17-29853LNA (ASO2), 18-63286LNA (ASO3), 17-63286LNA (ASO4), and 17-63458LNA (ASO10) are expected based on nucleotide sequence to have compatibility with the mouse Ube3a gene. To test target engagement of these ASOs in mice, Ube3a mRNA levels were measured in mouse brain cortex and hippocampus following CNS delivery using Quantigene RNA assay (N=2). As shown in FIG. 12, all ASOs tested show up to 3000 Ube3a knockdown in mouse brain regions post single bolus dose.

ANTISENSE COMPOUND SEQUENCES DESCRIBED HEREIN

| | Start | | | Start | |
|---|---|---|---|---|---|
| | position | | | position | |
| | with | | | with | |
| | respect | | | respect | |
| SEQ | to SEQ | | SEQ | to SEQ | |
| ID | ID NO: | | ID | ID NO: | |
| NO | 1 | Sequence | NO | 1 | Sequence |
| 2 | 480 | TGGTCGTAGTCGCCCT | 144 | 27092 | TCGCCAAACTTCTGAG |
| | | | | | GAGC |
| 3 | 482 | ACTGGTCGTAGTCGCC | 145 | 27093 | TTCGCCAAACTTCTGA |
| | | | | | GGAG |
| 4 | 29845 | CCAGTCTAGCTGCTAC | 146 | 29830 | GCTACCTTGATCTGAG |
| | | | | | CGTA |
| 5 | 29896 | ACATCAGGGTGATCAC | 147 | 29831 | TGCTACCTTGATCTGA |
| | | | | | GCGT |
| 6 | 29907 | GCCATTCGGTGACATC | 148 | 29836 | CTAGCTGCTACCTTGA |
| | | | | | TCTG |
| 7 | 29916 | CAAGCTGTGGCCATTC | 149 | 29838 | GTCTAGCTGCTACCTT |
| | | | | | GATC |
| 8 | 29917 | ACAAGCTGTGGCCATT | 150 | 29840 | CAGTCTAGCTGCTACC |
| | | | | | TTGA |
| 9 | 31774 | GCAAAACAAAAAATA | 151 | 29841 | CCAGTCTAGCTGCTAC |
| | | A | | | CTTG |
| 10 | 31920 | TAAGTACCCCAAGATT | 152 | 29846 | TCACACCAGTCTAGCT |
| | | | | | GCTA |
| 11 | 63309 | CCACAGCCCTCAGTTA | 153 | 29847 | GTCACACCAGTCTAGC |
| | | | | | TGCT |
| 12 | 63326 | TCGTGCAGGCTTCATT | 154 | 29852 | AGGTTGTCACACCAGT |
| | | | | | CTAG |
| 13 | 435 | CGTTCTCTTTCGCTGC | 155 | 29853 | CAGGTTGTCACACCAG |
| | | C | | | TCTA |
| 14 | 442 | ACCGCCTCGTTCTCTT | 156 | 29854 | ACAGGTTGTCACACCA |
| | | T | | | GTCT |
| 15 | 479 | TGGTCGTAGTCGCCCT | 157 | 29885 | GGTGATCACAGCTTTG |
| | | C | | | AGTC |
| 16 | 480 | CTGGTCGTAGTCGCCC | 158 | 29904 | GGCCATTCGGTGACAT |
| | | T | | | CAGG |
| 17 | 481 | ACTGGTCGTAGTCGCC | 159 | 29905 | TGGCCATTCGGTGACA |
| | | C | | | TCAG |
| 18 | 482 | CACTGGTCGTAGTCGC | 160 | 29906 | GTGGCCATTCGGTGAC |
| | | C | | | ATCA |
| 19 | 27055 | CTCTTACAGATTTTTA | 161 | 29907 | TGTGGCCATTCGGTGA |
| | | A | | | CATC |
| 20 | 27064 | AAAATCAGGCTCTTAC | 162 | 29909 | GCTGTGGCCATTCGGT |
| | | A | | | GACA |
| 21 | 27069 | ATTCTAAAATCAGGCT | 163 | 29910 | AGCTGTGGCCATTCGG |
| | | C | | | TGAC |
| 22 | 27100 | ATATTTCGCCAAACTT | 164 | 29911 | AAGCTGTGGCCATTCG |
| | | C | | | GTGA |
| 23 | 29817 | CGTAGGCTTAATAACT | 165 | 29912 | CAAGCTGTGGCCATTC |
| | | C | | | GGTG |
| 24 | 29823 | TCTGAGCGTAGGCTTA | 166 | 29915 | TTACAAGCTGTGGCCA |
| | | A | | | TTCG |
| 25 | 29839 | CTAGCTGCTACCTTGA | 167 | 33523 | CAGACTGAGGTTCTCC |
| | | T | | | TGAT |
| 26 | 29855 | AGGTTGTCACACCAGT | 168 | 33524 | TCAGACTGAGGTTCTC |
| | | C | | | CTGA |
| 27 | 29858 | AACAGGTTGTCACACC | 169 | 33525 | GTCAGACTGAGGTTCT |
| | | A | | | CCTG |
| 28 | 29859 | AAACAGGTTGTCACAC | 170 | 33526 | CGTCAGACTGAGGTTC |
| | | C | | | TCCT |
| 29 | 29879 | AGCTTTGAGTCACTGA | 171 | 33528 | GTCGTCAGACTGAGGT |
| | | T | | | TCTC |
| 30 | 29906 | GCCATTCGGTGACATC | 172 | 33529 | TGTCGTCAGACTGAGG |
| | | A | | | TTCT |
| 31 | 29907 | GGCCATTCGGTGACAT | 173 | 33530 | ATGTCGTCAGACTGAG |
| | | C | | | GTTC |
| 32 | 29916 | ACAAGCTGTGGCCATT | 174 | 33532 | CAATGTCGTCAGACTG |
| | | C | | | AGGT |
| 33 | 29917 | TACAAGCTGTGGCCAT | 175 | 33536 | GCTTCAATGTCGTCAG |
| | | T | | | ACTG |
| 34 | 29918 | TTACAAGCTGTGGCCA | 176 | 33540 | GCTAGCTTCAATGTCG |
| | | T | | | TCAG |
| 35 | 29919 | TTTACAAGCTGTGGCC | 177 | 33541 | GGCTAGCTTCAATGTC |
| | | A | | | GTCA |
| 36 | 29920 | TTTTACAAGCTGTGGC | 178 | 33542 | CGGCTAGCTTCAATGT |
| | | C | | | CGTC |
| 37 | 33529 | CGTCAGACTGAGGTTC | 179 | 33543 | TCGGCTAGCTTCAATG |
| | | T | | | TCGT |
| 38 | 33531 | GTCGTCAGACTGAGGT | 180 | 33544 | TTCGGCTAGCTTCAAT |
| | | T | | | GTCG |
| 39 | 33541 | TAGCTTCAATGTCGTC | 181 | 63263 | TGCTTTGCAGCTGCTC |
| | | A | | | GCTT |
| 40 | 33545 | CGGCTAGCTTCAATGT | 182 | 63264 | ATGCTTTGCAGCTGCT |
| | | C | | | CGCT |
| 41 | 33548 | ATTCGGCTAGCTTCAA | 183 | 63265 | GATGCTTTGCAGCTGC |
| | | T | | | TCGC |
| 42 | 33549 | CATTCGGCTAGCTTCA | 184 | 63266 | AGATGCTTTGCAGCTG |
| | | A | | | CTCG |
| 43 | 63262 | TTGCAGCTGCTCGCTT | 185 | 63288 | CTGGTGGTAGTAGCGT |
| | | C | | | TCTA |
| 44 | 63264 | CTTTGCAGCTGCTCGC | 186 | 63289 | ACTGGTGGTAGTAGCG |
| | | T | | | TTCT |
| 45 | 63289 | GGTGGTAGTAGCGTTC | 187 | 63290 | AACTGGTGGTAGTAGC |
| | | T | | | GTTC |
| 46 | 63290 | TGGTGGTAGTAGCGTT | 188 | 63293 | GTTAACTGGTGGTAGT |
| | | C | | | AGCG |
| 47 | 63291 | CTGGTGGTAGTAGCGT | 189 | 63301 | AGCCCTCAGTTAACTG |
| | | T | | | GTGG |
| 48 | 63306 | ACAGCCCTCAGTTAAC | 190 | 63302 | CAGCCCTCAGTTAACT |
| | | T | | | GGTG |
| 49 | 63306 | ACAGCCCTCAGTTAAC | 191 | 63303 | ACAGCCCTCAGTTAAC |
| | SNPE | A | | | TGGT |
| 50 | 63306 | ACAGCCCTAAGTTAAC | 192 | 63304 | CACAGCCCTCAGTTAA |
| | SNPM | T | | | CTGG |
| 51 | 63307 | CACAGCCCTCAGTTAA | 193 | 63305 | CCACAGCCCTCAGTTA |
| | | C | | | ACTG |
| 52 | 63308 | CCACAGCCCTCAGTTA | 194 | 63306 | TCCACAGCCCTCAGTT |
| | | A | | | AACT |
| 53 | 63309 | TCCACAGCCCTCAGTT | 195 | 63307 | TTCCACAGCCCTCAGT |
| | | A | | | TAAC |
| 54 | 63320 | CAGGCTTCATTTCCAC | 196 | 63307 | TTCCACAGCCCTCAGT |
| | | A | | SNPE | TGAC |
| 55 | 63321 | GCAGGCTTCATTTCCA | 197 | 63307 | TTCCACAGCGCTCAGT |
| | | C | | SNPM | TAAC |
| 56 | 63323 | GTGCAGGCTTCATTTC | 198 | 63310 | CATTTCCACAGCCCTC |
| | | C | | | AGTT |
| 57 | 63324 | CGTGCAGGCTTCATTT | 199 | 63311 | TCATTTCCACAGCCCT |
| | | C | | | CAGT |
| 58 | 63325 | TCGTGCAGGCTTCATT | 200 | 63317 | CAGGCTTCATTTCCAC |
| | | T | | | AGCC |
| 59 | 63326 | TTCGTGCAGGCTTCAT | 201 | 63318 | GCAGGCTTCATTTCCA |
| | | T | | | CAGC |
| 60 | 63327 | ATTCGTGCAGGCTTCA | 202 | 63319 | TGCAGGCTTCATTTCC |
| | | T | | | ACAG |
| 61 | 63340 | AAGCACAAAACTCATT | 203 | 63320 | GTGCAGGCTTCATTTC |
| | | C | | | CACA |
| 62 | 63348 | TGGACAGGAAGCACA | 204 | 63321 | CGTGCAGGCTTCATTT |
| | | AA | | | CCAC |
| 63 | 63351 | AGTTGGACAGGAAGC | 205 | 63322 | TCGTGCAGGCTTCATT |
| | | AC | | | TCCA |
| 64 | 63352 | AAGTTGGACAGGAAG | 206 | 63323 | TTCGTGCAGGCTTCAT |
| | | CA | | | TTCC |
| 65 | 63364 | CCATACGAAGAAAAG | 207 | 63327 | CTCATTCGTGCAGGCT |
| | | TT | | | TCAT |
| 66 | 63410 | TTAATCTTATAAAGCT | 208 | 63332 | CAAAACTCATTCGTGC |
| | | C | | | AGGC |
| 67 | 63457 | CTGAGCTTGCTCCTTT | 209 | 63344 | GGACAGGAAGCACAA |
| | | C | | | AACTC |
| 68 | 63458 | GCTGAGCTTGCTCCTT | 210 | 63411 | TGCATTAATCTTATAA |
| | | T | | | AGCT |
| 69 | 63463 | GGTAAGCTGAGCTTGC | 211 | 63450 | GCTTGCTCCTTTCTTG |
| | | T | | | GAGG |
| 70 | 63467 | TCAAGGTAAGCTGAG | 212 | 63451 | AGCTTGCTCCTTTCTT |
| | | CT | | | GGAG |
| 71 | 67212 | GTTAAGTAAGTCACAT | 213 | 63452 | GAGCTTGCTCCTTTCT |
| | | C | | | TGGA |
| 72 | 67242 | AATTCAAGAATTTCAT | 214 | 63453 | TGAGCTTGCTCCTTTC |
| | | A | | | TTGG |
| 73 | 67260 | TCCTCTCTTTCTCTACA | 215 | 63454 | CTGAGCTTGCTCCTTT |
| | | | | | CTTG |
| 74 | 67307 | CAGCACTAGAAAAAA | 216 | 63455 | GCTGAGCTTGCTCCTT |
| | | CT | | | TCTT |
| 75 | 67312 | TGCCTCAGCACTAGAA | 217 | 63456 | AGCTGAGCTTGCTCCT |
| | | A | | | TTCT |
| 76 | 435 | TCGTTCTCTTTCGCTG | 218 | 63457 | AAGCTGAGCTTGCTCC |
| | | CC | | | TTTC |
| 77 | 438 | GCCTCGTTCTCTTTCG | 219 | 63459 | GTAAGCTGAGCTTGCT |
| | | CT | | | CCTT |
| 78 | 480 | ACTGGTCGTAGTCGCC | 220 | 63460 | GGTAAGCTGAGCTTGC |
| | | CT | | | TCCT |
| 79 | 482 | TCACTGGTCGTAGTCG | 221 | 63461 | AGGTAAGCTGAGCTTG |
| | | CC | | | CTCC |
| 80 | 861 | CCTGTCTGGGGCCCCG | 222 | 63462 | AAGGTAAGCTGAGCTT |
| | | GC | | | GCTC |
| 81 | 27091 | CCAAACTTCTGAGGAG | 223 | 63463 | CAAGGTAAGCTGAGCT |
| | | CT | | | TGCT |
| 82 | 29838 | CTAGCTGCTACCTTGA | 224 | 63464 | TCAAGGTAAGCTGAGC |
| | | TC | | | TTGC |
| 83 | 29854 | AGGTTGTCACACCAGT | 225 | 63478 | CACCTTTCGAGTTCTC |
| | | CT | | | AAGG |
| 84 | 29906 | GGCCATTCGGTGACAT | 226 | 63479 | GCACCTTTCGAGTTCT |
| | | CA | | | CAAG |
| 85 | 29907 | TGGCCATTCGGTGACA | 227 | 67202 | AAGTCACATCTAGAAA |
| | | TC | | | ATCA |
| 86 | 29916 | TACAAGCTGTGGCCAT | 228 | 67248 | TCTCTACATAATTCAA |
| | | TC | | | GAAT |
| 87 | 29920 | CTTTTACAAGCTGTGG | 229 | 67296 | GAAAAAACTCTTCCAA |
| | | CC | | | TAAC |
| 88 | 33523 | GACTGAGGTTCTCCTG | 230 | 67319 | TCTGTACCAATGCCTC |
| | | AT | | | AGCA |
| 89 | 33526 | TCAGACTGAGGTTCTC | 231 | 67320 | CTCTGTACCAATGCCT |
| | | CT | | | CAGC |
| 90 | 33526 | TCAGACTGAGGTTCTC | 232 | 67321 | GCTCTGTACCAATGCC |
| | SNPE | CC | | | TCAG |
| 91 | 33526 | TCAGACTGACGTTCTC | 233 | 67323 | AAGCTCTGTACCAATG |
| | SNPM | CT | | | CCTC |
| 92 | 33528 | CGTCAGACTGAGGTTC | 234 | 29818 | GCGTAGGCTTAATAAC |
| | | TC | | | T |
| 93 | 33530 | GTCGTCAGACTGAGGT | 235 | 29819 | AGCGTAGGCTTAATAA |
| | | TC | | | C |
| 94 | 33531 | TGTCGTCAGACTGAGG | 236 | 29820 | GAGCGTAGGCTTAATA |
| | | TT | | | A |
| 95 | 33538 | GCTTCAATGTCGTCAG | 237 | 29821 | TGAGCGTAGGCTTAAT |
| | | AC | | | A |
| 96 | 33542 | GCTAGCTTCAATGTCG | 238 | 29822 | CTGAGCGTAGGCTTAA |
| | | TC | | | T |
| 97 | 33545 | TCGGCTAGCTTCAATG | 239 | 63274 | CTATTAGATGCTTTGC |
| | | TC | | | A |
| 98 | 63262 | TTTGCAGCTGCTCGCT | 240 | 63278 | CGTTCTATTAGATGCT |
| | | TC | | | T |
| 99 | 63263 | CTTTGCAGCTGCTCGC | 241 | 63279 | GCGTTCTATTAGATGC |
| | | TT | | | T |
| 100 | 63264 | GCTTTGCAGCTGCTCG | 242 | 63282 | GTAGCGTTCTATTAGA |
| | | CT | | | T |
| 101 | 63268 | AGATGCTTTGCAGCTG | 243 | 63285 | GTAGTAGCGTTCTATT |
| | | CT | | | A |
| 102 | 63290 | CTGGTGGTAGTAGCGT | 244 | 63286 | GGTAGTAGCGTTCTAT |
| | | TC | | | T |
| 103 | 63291 | ACTGGTGGTAGTAGCG | 245 | 63287 | TGGTAGTAGCGTTCTA |
| | | TT | | | T |
| 104 | 63306 | CACAGCCCTCAGTTAA | 246 | 63288 | GTGGTAGTAGCGTTCT |
| | | CT | | | A |
| 105 | 63307 | CCACAGCCCTCAGTTA | 247 | 63296 | GTTAACTGGTGGTAGT |
| | | AC | | | A |
| 106 | 63308 | TCCACAGCCCTCAGTT | 248 | 63299 | TCAGTTAACTGGTGGT |
| | | AA | | | A |
| 107 | 63309 | TTCCACAGCCCTCAGT | 249 | 63342 | GGAAGCACAAAACTC |
| | | TA | | | AT |
| 108 | 63320 | GCAGGCTTCATTTCCA | 250 | 63343 | AGGAAGCACAAAACT |
| | | CA | | | CA |
| 109 | 63321 | TGCAGGCTTCATTTCC | 251 | 63346 | GACAGGAAGCACAAA |
| | | AC | | | AC |
| 110 | 63322 | GTGCAGGCTTCATTTC | 252 | 63398 | AGCTCGAGGGCTTTAA |
| | | CA | | | T |
| 111 | 63323 | CGTGCAGGCTTCATTT | 253 | 63399 | AAGCTCGAGGGCTTTA |
| | | CC | | | A |
| 112 | 63324 | TCGTGCAGGCTTCATT | 254 | 63401 | TAAAGCTCGAGGGCTT |
| | | TC | | | T |
| 113 | 63327 | CATTCGTGCAGGCTTC | 255 | 63402 | ATAAAGCTCGAGGGCT |
| | | AT | | | T |
| 114 | 63346 | GGACAGGAAGCACAA | 256 | 63437 | GAGGGATGAGGATCA |
| | | AAC | | | CA |
| 115 | 63350 | AGTTGGACAGGAAGC | 257 | 63441 | CTTGGAGGGATGAGG |
| | | ACA | | | AT |
| 116 | 63351 | AAGTTGGACAGGAAG | 258 | 29847 | ACACCAGTCTAGCTGC |
| | | CAC | | | T |
| 117 | 63456 | CTGAGCTTGCTCCTTT | 259 | 29853 | GTTGTCACACCAGTCT |
| | | CT | | | A |
| 118 | 63457 | GCTGAGCTTGCTCCTT | 260 | 29854 | GGTTGTCACACCAGTC |
| | | TC | | | T |
| 119 | 63458 | AGCTGAGCTTGCTCCT | 261 | 29859 | AAAACAGGTTGTCACA |
| | | TT | | | CC |
| 120 | 63460 | TAAGCTGAGCTTGCTC | 262 | 29860 | AAAAACAGGTTGTCAC |
| | | CT | | | AC |
| 121 | 63462 | GGTAAGCTGAGCTTGC | 263 | 63274 | TCTATTAGATGCTTTG |
| | | TC | | | CA |
| 122 | 63463 | AGGTAAGCTGAGCTTG | 264 | 63278 | GCGTTCTATTAGATGC |
| | | CT | | | TT |
| 123 | 64667 | CCCCCGCAGCTGGCTG | 265 | 63279 | AGCGTTCTATTAGATG |
| | | CA | | | CT |
| 124 | 64668 | TCCCCCGCAGCTGGCT | 266 | 63282 | AGTAGCGTTCTATTAG |
| | | GC | | | AT |
| 125 | 431 | GTTCTCTTTCGCTGCC | 267 | 63285 | GGTAGTAGCGTTCTAT |
| | | GCCA | | | TA |
| 126 | 433 | TCGTTCTCTTTCGCTG | 268 | 63286 | TGGTAGTAGCGTTCTA |
| | | CCGC | | | TT |
| 127 | 434 | CTCGTTCTCTTTCGCT | 269 | 63287 | GTGGTAGTAGCGTTCT |
| | | GCCG | | | AT |
| 128 | 436 | GCCTCGTTCTCTTTCG | 270 | 63325 | TTCGTGCAGGCTTCAT |
| | | CTGC | | | TT |
| 129 | 437 | CGCCTCGTTCTCTTTC | 271 | 63326 | ATTCGTGCAGGCTTCA |
| | | GCTG | | | TT |
| 130 | 439 | ACCGCCTCGTTCTCTT | 272 | 63437 | GGAGGGATGAGGATC |
| | | TCGC | | | ACA |
| 131 | 440 | CACCGCCTCGTTCTCT | 273 | 63439 | TTGGAGGGATGAGGA |
| | | TTCG | | | TCA |
| 132 | 441 | CCACCGCCTCGTTCTC | 274 | 63440 | CTTGGAGGGATGAGG |
| | | TTTC | | | ATC |
| 133 | 442 | GCCACCGCCTCGTTCT | 275 | 63447 | CTCCTTTCTTGGAGGG |
| | | CTTT | | | AT |
| 134 | 480 | TCACTGGTCGTAGTCG | 276 | 424 | TTCGCTGCCGCCACCG |
| | | CCCT | | | CCGG |
| 135 | 27066 | ATTCTAAAATCAGGCT | 277 | 425 | TTTCGCTGCCGCCACC |
| | | CTTA | | | GCCG |
| 136 | 27073 | CTGGTGAATTCTAAAA | 278 | 426 | CTTTCGCTGCCGCCAC |
| | | TCAG | | | CGCC |
| 137 | 27084 | CTTCTGAGGAGCTGGT | 279 | 432 | CGTTCTCTTTCGCTGC |
| | | GAAT | | | CGCC |
| 138 | 27085 | ACTTCTGAGGAGCTGG | 280 | SCRA1 | GATTACAGATTACACA |
| | | TGAA | | | T |
| 139 | 27086 | AACTTCTGAGGAGCTG | 281 | SCRA2 | CATCATCATCATCATC |
| | | GTGA | | | AT |
| 140 | 27087 | AAACTTCTGAGGAGCT | 282 | SCRA3 | ACATCATCATCATCAT |
| | | GGTG | | | CATC |
| 141 | 27088 | CAAACTTCTGAGGAGC | 283 | CTRL1 | GGATTCAACTGCTGTC |
| | | TGGT | | | CTTG |
| 142 | 27090 | GCCAAACTTCTGAGGA | 284 | CTRL2 | TGAGCTATCACCTATC |
| | | GCTG | | | CTTG |
| 143 | 27091 | CGCCAAACTTCTGAGG | | | |
| | | AGCT | | | |

1. An antisense oligonucleotide (ASO) comprising a nucleotide sequence selected from SEQ ID NO: 267 (GGTAGTAGCGTTCTATTA), SEQ ID NO: 244 (GGTAGTAGCGTTCTATT), SEQ ID NO: 68 (GCTGAGCTTGCTCCTTT), SEQ ID NO: 111 (CGTGCAGGCTTCATTTCC), SEQ ID NO: 268 (TGGTAGTAGCGTTCTATT), SEQ ID NO: 259 (GTTGTCACACCAGTCTA), SEQ ID NO: 58 (TCGTGCAGGCTTCATTT), SEQ ID NO: 57 (CGTGCAGGCTTCATTTC), SEQ ID NO: 12 (TCGTGCAGGCTTCATT), SEQ ID NO: 73 (TCCTCTCTTTCTCTACA), SEQ ID NO: 46 (TGGTGGTAGTAGCGTTC), and SEQ ID NO: 47 (CTGGTGGTAGTAGCGTT);
wherein the ASO comprises, in 5′ to 3′ order, a 5′ segment, a gap segment, and a 3′ segment; and
wherein the ASO comprises one or more of at least one phosphorothioate internucleoside linkage; at least one nucleoside comprising a modified sugar; and at least one nucleoside comprising a modified nucleobase.

2. (canceled)

3. (canceled)

4. The ASO of claim 1, wherein the 3′ segment comprises 2-5 linked nucleosides and the 5′ segment comprises 2-5 linked nucleosides.

5. (canceled)

6. The ASO of claim 1, wherein at least one nucleoside of the 5′ segment and at least one nucleoside of the 3′ segment comprises a modified sugar.

7. (canceled)

8. (canceled)

9. The ASO of claim 6, wherein the modified sugar is selected from the group consisting of: 2′-O(CH2)2OCH3 (MOE); 4′-(CH2)-O-2′ (LNA); 4′-(CH2)2-O-2′ (ENA); and 4′-CH(CH3)-O-2′ (cEt).

10. The ASO of claim 1, wherein each internucleoside linkage is a phosphorothioate internucleoside linkage.

11. The ASO of claim 1, wherein at least one cytosine nucleobase in the ASO is a 5-methylcytosine nucleobase.

12. The ASO of claim 1, comprising a 3-10-3 LNA-DNA-LNA gapmer, wherein all internucleoside linkages are phosphorothioate internucleoside linkages.

13. The ASO of claim 1, comprising a 3-11-3 LNA-DNA-LNA gapmer, wherein all internucleoside linkages are phosphorothioate internucleoside linkages.

14. The ASO of claim 1, comprising a 3-12-3 LNA-DNA-LNA gapmer, wherein all internucleoside linkages are phosphorothioate internucleoside linkages.

15. The ASO of claim 1, comprising a 4-11-5 MOE-DNA-MOE gapmer, wherein all internucleoside linkages are phosphorothioate internucleoside linkages.

16. An antisense oligonucleotide (ASO), wherein the ASO is a gapmer according to Compound ID No. 16-63326LNA, 17-29823LNA, 17-29855LNA, 17-29858LNA, 17-63264LNA, 17-63289LNA, 17-63290LNA, 17-63291LNA, 17-63324LNA, 17-63325LNA, 17-63326LNA, 17-63327LNA, 17-63458LNA, 17-67260LNA, 18-435LNA, 18-29854LNA, 18-63323LNA, 18-63458LNA, 17-63278LNA, 17-63286LNA, 17-63287LNA, 17-63288LNA, 17-63437LNA, 17-29853LNA, 18-63282LNA, 18-63285LNA, 18-63286LNA, or 18-63325LNA.

17-26. (canceled)

27. The ASO of claim 1, comprising a 2′OMe-modified nucleoside at the second nucleotide position from the 5′ end in the gap segment.

28. A composition comprising the ASO of claim 1 or a salt thereof and a pharmaceutically acceptable carrier.

29. (canceled)

30. A method of reducing the level of a UBE3A RNA or UBE3A protein in a cell, comprising contacting the cell with the ASO of claim 1, thereby reducing the level of the UBE3A RNA or UBE3A protein in the cell.

31. (canceled)

32. The method of claim 30, wherein the cell is a central nervous system neuron.

33. The method of claim 30, wherein the cell is triploid or tetraploid for the UBE3A gene.

34. (canceled)

35. The method of claim 30, wherein the cell is in vitro.

36-38. (canceled)

39. A method of treating, preventing, or ameliorating a disease associated with overexpression of UBE3A in central nervous system neurons in a subject, comprising administering to the subject the ASO of claim 1, thereby treating, preventing, or ameliorating the disease.

40. The method of claim 39, wherein the disease is Dup15q syndrome.

41. The method of claim 39, wherein the subject has an idic(15) genotype.

42-49. (canceled)
